SEQUENCE LISTING (25 sequences)

SEQ ID NO: 1 - protein, 776 aa, unidentified
  source 1..776: note = synthetic sequence
MKRHLNTCYRLVWNHMTGAFVVASELARARGKRGGVAVALSLAAVTSLPVLAGGGHVVKWGYVGKIGPAHWGDLAHEYFMCKVGKNQSPVDINSSVTIEAQLEPINFHYRDQISGEIVNNGHTIMVVPKEDNYIVVDGKKFHLKQFHFHSPSEHTVEGKYYLLELHFVHQADDGQLAVIGVVFDRGAEHPEIAKLWKEAPEHEGKKELKSLVNMQALLPENLDYYRYSGSLTTPPCSEGVIWLFLKNPLQISEAQAEKFKKIMGFENNRPVQPVNARKILKGSSSGSSPTNVTLASGATWNIPDNATVQSVVDDLSHAGQIHFTSTRTGKFVPATLKVKNLNGQNGTISLRVRPDMAQNNADRLVIDGGRATGKTILNLVNAGNSASGLATSGKGIQVVEAINGATTEEGAFVQGNRLQAGAFNYSLNRDSDESWYLRSENAYRAEVPLYASMLTQAMDYDRIVAGSRSHQTGVNGENNSVRLSIQGGHLGHDNNGGIARGATPESSGSYGFVRLEGDLMRTEVAGMSVTAGVYGAAGHSSVDVKDDDGSRAGTVRDDAGSLGGYLNLVHTSSGLWADIVAQGTRHSMKASSDNNDFRARGWGWLGSLETGLPFSITDNLMLEPQLQYTWQGLSLDDGKDNAGYVKFGHGSAQHVRAGFRLGSHNDMTFGEGTSSRAPLRDSAKHSVSELPVNWWVQPSVIRTFSSRGDMRVGTSTAGSGMTFSPSQNGTSLDLQAGLEARVRENITLGVQAGYAHSVSGSSAEGYNGQATLNVTF

SEQ ID NO: 2 - protein, 751 aa, unidentified
  source 1..751: note = synthetic sequence
MKRLFSALLLAPAIAGVAAGAANANGLGGGHVVKWGYVGKIGPAHWGDLAHEYFMCKVGKNQSPVDINSSVTIEAQLEPINFHYRDQISGEIVNNGHTIMVVPKEDNYIVVDGKKFHLKQFHFHSPSEHTVEGKYYLLELHFVHQADDGQLAVIGVVFDRGAEHPEIAKLWKEAPEHEGKKELKSLVNMQALLPENLDYYRYSGSLTTPPCSEGVIWLFLKNPLQISEAQAEKFKKIMGFENNRPVQPVNARKILKGSSSGSSPTNVTLASGATWNIPDNATVQSVVDDLSHAGQIHFTSTRTGKFVPATLKVKNLNGQNGTISLRVRPDMAQNNADRLVIDGGRATGKTILNLVNAGNSASGLATSGKGIQVVEAINGATTEEGAFVQGNRLQAGAFNYSLNRDSDESWYLRSENAYRAEVPLYASMLTQAMDYDRIVAGSRSHQTGVNGENNSVRLSIQGGHLGHDNNGGIARGATPESSGSYGFVRLEGDLMRTEVAGMSVTAGVYGAAGHSSVDVKDDDGSRAGTVRDDAGSLGGYLNLVHTSSGLWADIVAQGTRHSMKASSDNNDFRARGWGWLGSLETGLPFSITDNLMLEPQLQYTWQGLSLDDGKDNAGYVKFGHGSAQHVRAGFRLGSHNDMTFGEGTSSRAPLRDSAKHSVSELPVNWWVQPSVIRTFSSRGDMRVGTSTAGSGMTFSPSQNGTSLDLQAGLEARVRENITLGVQAGYAHSVSGSSAEGYNGQATLNVTF

SEQ ID NO: 3 - protein, 1359 aa, unidentified
  source 1..1359: note = synthetic sequence
MNRIYSLRYSAVARGFIAVSEFARKCVHKSVRRLCFPVLLLIPVLFSAGSLAGGGGHVVKWGYVGKIGPAHWGDLAHEYFMCKVGKNQSPVDINSSVTIEAQLEPINFHYRDQISGEIVNNGHTIMVVPKEDNYIVVDGKKFHLKQFHFHSPSEHTVEGKYYLLELHFVHQADDGQLAVIGVVFDRGAEHPEIAKLWKEAPEHEGKKELKSLVNMQALLPENLDYYRYSGSLTTPPCSEGVIWLFLKNPLQISEAQAEKFKKIMGFENNRPVQPVNARKILKGSSSGSSNDAPVTFRTSEGGALEWSFNSSTGAGALTQGTTTYAMHGQQGNDLNAGKNLIFQGQNGQINLKDSVSQGAGSLTFRDNYTVTTSNGSTWTGAGIVVDNGVSVNWQVNGVKGDNLHKIGEGTLTVQGTGINEGGLKVGDGKVVLNQQADNKGQVQAFSSVNIASGRPTVVLTDERQVNPDTVSWGYRGGTLDVNGNSLTFHQLKAADYGAVLANNVDKRATITLDYALRADKVALNGWSESGKGTAGNLYKYNNPYTNTTDYFILKQSTYGYFPTDQSSNATWEFVGHSQGDAQKLVADRFNTAGYLFHGQLKGNLNVDNRLPEGVTGALVMDGAADISGTFTQENGRLTLQGHPVIHAYNTQSVADKLAASGDHSVLTQPTSFSQEDWENRSFTFDRLSLKNTDFGLGRNATLNTTIQADNSSVTLGDSRVFIDKNDGQGTAFTLEEGTSVATKDADKSVFNGTVNLDNQSVLNINDIFNGGIQANNSTVNISSDSAVLGNSTLTSTALNLNKGANALASQSFVSDGPVNISDATLSLNSRPDEVSHTLLPVYDYAGSWNLKGDDARLNVGPYSMLSGNINVQDKGTVTLGGEGELSPDLTLQNQMLYSLFNGYRNIWSGSLNAPDATVSMTDTQWSMNGNSTAGNMKLNRTIVGFNGGTSPFTTLTTDNLDAVQSAFVMRTDLNKADKLVINKSATGHDNSIWVNFLKKPSNKDTLDIPLVSAPEATADNLFRASTRVVGFSDVTPILSVRKEDGKKEWVLDGYQVARNDGQGKAAATFMHISYNNFITEVGSLNKRMGDLRDINGEAGTWVRLLNGSGSADGGFTDHYTLLQMGADRKHELGSMDLFTGVMATYTDTDASADLYSGKTKSWGGGFYASGLFRSGAYFDVIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDSRMLYGVGLNARFGDNTRLGLEVERSAFGKYNTDDAINANIRYSF

SEQ ID NO: 4 - protein, 611 aa, unidentified
  source 1..611: note = synthetic sequence
MKRLFSALLLAPAIAGVAAGAANANGLSTEQLQKIDAVTPNGITSGQITSITELSDVKPTDWAYQALQSLVERYGCIVGYPDRTYRGSRPLSRYEFAAGLNACLDKVIEFAASKEDLDTLKRLTEEFQAELATLRGRVDSLEARVKELEATRFSTTTKLQGEVIFSLDAVANTAGNERNQDGAVSFGNRVSLNLNTSFTGKDLLLTRLRARNIETIQQRLSPGFNPSGSRLDYDGTGSPGVPNSANTFFLDKLLYRFPVGDVSFTVGTAGVQPQDYGLSDATFFSGPANTKAFKYVGAGVYADTRDADTAGVGFNWKASKNFSFQAGYINRNSADVSTVNSGGVFGFTPTGTGTNSWDVNAQVKYQTDNNKFRVALAYALRNGGGHVVKWGYVGKIGPAHWGDLAHEYFMCKVGKNQSPVDINSSVTIEAQLEPINFHYRDQISGEIVNNGHTIMVVPKEDNYIVVDGKKFHLKQFHFHSPSEHTVEGKYYLLELHFVHQADDGQLAVIGVVFDRGAEHPEIAKLWKEAPEHEGKKELKSLVNMQALLPENLDYYRYSGSLTTPPCSEGVIWLFLKNPLQISEAQAEKFKKIMGFENNRPVQPVNARKILK

SEQ ID NO: 5 - protein, 392 aa, Unidentified
  source 1..392: note = synthetic sequence
MTKLKLLALGVLIATSAGVAHAEGKFSLGAGVGVVEHPYKDYDTDVYPVPVINYEGDNFWFRGLGGGYYLWNDATDKLSITAYWSPLYFKAKDSGDHQMRHLDDRKSTMMAGLSYAHFTQYGYLRTTLAGDTLDNSNGIVGGGGSGGSGGGGSGGGGSGGGGSGGGHVVKWGYVGKIGPAHWGDLAHEYFMCKVGKNQSPVDINSSVTIEAQLEPINFHYRDQISGEIVNNGHTIMVVPKEDNYIVVDGKKFHLKQFHFHSPSEHTVEGKYYLLELHFVHQADDGQLAVIGVVFDRGAEHPEIAKLWKEAPEHEGKKELKSLVNMQALLPENLDYYRYSGSLTTPPCSEGVIWLFLKNPLQISEAQAEKFKKIMGFENNRPVQPVNARKILK

SEQ ID NO: 6 - protein, 473 aa, Unidentified
  source 1..473: note = synthetic sequence
MKYLLPTAAAGLLLLAAQPAMAMTLDKALVLRTCANNMADHCGLIWPASGTVESRYWQSTRRHENGLVGLLWGAGTSAFLSVHADARWIVCEVAVADIISLEEPGMVKFPRAEVVHVGDRISASHFISARQADPASTSTSTSTSTLTPMPTAIPTPMPAVASVTLPVAEQARHEVFDVASVSAAAAPVNTLPVTTPQNLQTATYGSTLSGDNHSRLIAGYGSNETAGNHSDLIGGGGSGGGHVVKWGYVGKIGPAHWGDLAHEYFMCKVGKNQSPVDINSSVTIEAQLEPINFHYRDQISGEIVNNGHTIMVVPKEDNYIVVDGKKFHLKQFHFHSPSEHTVEGKYYLLELHFVHQADDGQLAVIGVVFDRGAEHPEIAKLWKEAPEHEGKKELKSLVNMQALLPENLDYYRYSGSLTTPPCSEGVIWLFLKNPLQISEAQAEKFKKIMGFENNRPVQPVNARKILKHHHHHH

SEQ ID NO: 7 - protein, 229 aa, Unidentified
  source 1..229: note = synthetic sequence
GGGHVVKWGYVGKIGPAHWGDLAHEYFMCKVGKNQSPVDINSSVTIEAQLEPINFHYRDQISGEIVNNGHTIMVVPKEDNYIVVDGKKFHLKQFHFHSPSEHTVEGKYYLLELHFVHQADDGQLAVIGVVFDRGAEHPEIAKLWKEAPEHEGKKELKSLVNMQALLPENLDYYRYSGSLTTPPCSEGVIWLFLKNPLQISEAQAEKFKKIMGFENNRPVQPVNARKILK

SEQ ID NO: 8 - protein, 488 aa, unidentified
  source 1..488: note = synthetic sequence
PTNVTLASGATWNIPDNATVQSVVDDLSHAGQIHFTSTRTGKFVPATLKVKNLNGQNGTISLRVRPDMAQNNADRLVIDGGRATGKTILNLVNAGNSASGLATSGKGIQVVEAINGATTEEGAFVQGNRLQAGAFNYSLNRDSDESWYLRSENAYRAEVPLYASMLTQAMDYDRIVAGSRSHQTGVNGENNSVRLSIQGGHLGHDNNGGIARGATPESSGSYGFVRLEGDLMRTEVAGMSVTAGVYGAAGHSSVDVKDDDGSRAGTVRDDAGSLGGYLNLVHTSSGLWADIVAQGTRHSMKASSDNNDFRARGWGWLGSLETGLPFSITDNLMLEPQLQYTWQGLSLDDGKDNAGYVKFGHGSAQHVRAGFRLGSHNDMTFGEGTSSRAPLRDSAKHSVSELPVNWWVQPSVIRTFSSRGDMRVGTSTAGSGMTFSPSQNGTSLDLQAGLEARVRENITLGVQAGYAHSVSGSSAEGYNGQATLNVTF

SEQ ID NO: 9 - protein, 1070 aa, Unidentified
  source 1..1070: note = synthetic sequence
NDAPVTFRTSEGGALEWSFNSSTGAGALTQGTTTYAMHGQQGNDLNAGKNLIFQGQNGQINLKDSVSQGAGSLTFRDNYTVTTSNGSTWTGAGIVVDNGVSVNWQVNGVKGDNLHKIGEGTLTVQGTGINEGGLKVGDGKVVLNQQADNKGQVQAFSSVNIASGRPTVVLTDERQVNPDTVSWGYRGGTLDVNGNSLTFHQLKAADYGAVLANNVDKRATITLDYALRADKVALNGWSESGKGTAGNLYKYNNPYTNTTDYFILKQSTYGYFPTDQSSNATWEFVGHSQGDAQKLVADRFNTAGYLFHGQLKGNLNVDNRLPEGVTGALVMDGAADISGTFTQENGRLTLQGHPVIHAYNTQSVADKLAASGDHSVLTQPTSFSQEDWENRSFTFDRLSLKNTDFGLGRNATLNTTIQADNSSVTLGDSRVFIDKNDGQGTAFTLEEGTSVATKDADKSVFNGTVNLDNQSVLNINDIFNGGIQANNSTVNISSDSAVLGNSTLTSTALNLNKGANALASQSFVSDGPVNISDATLSLNSRPDEVSHTLLPVYDYAGSWNLKGDDARLNVGPYSMLSGNINVQDKGTVTLGGEGELSPDLTLQNQMLYSLFNGYRNIWSGSLNAPDATVSMTDTQWSMNGNSTAGNMKLNRTIVGFNGGTSPFTTLTTDNLDAVQSAFVMRTDLNKADKLVINKSATGHDNSIWVNFLKKPSNKDTLDIPLVSAPEATADNLFRASTRVVGFSDVTPILSVRKEDGKKEWVLDGYQVARNDGQGKAAATFMHISYNNFITEVGSLNKRMGDLRDINGEAGTWVRLLNGSGSADGGFTDHYTLLQMGADRKHELGSMDLFTGVMATYTDTDASADLYSGKTKSWGGGFYASGLFRSGAYFDVIAKYIHNENKYDLNFAGAGKQNFRSHSLYAGAEVGYRYHLTDTTFVEPQAELVWGRLQGQTFNWNDSGMDVSMRRNSVNPLVGRTGVVSGKTFSGKDWSLTARAGLHYEFDLTDSADVHLKDAAGEHQINGRKDSRMLYGVGLNARFGDNTRLGLEVERSAFGKYNTDDAINANIRYSF

SEQ ID NO: 10 - protein, 382 aa, Unidentified
  source 1..382: note = synthetic sequence
MKRLFSALLLAPAIAGVAAGAANANGLSTEQLQKIDAVTPNGITSGQITSITELSDVKPTDWAYQALQSLVERYGCIVGYPDRTYRGSRPLSRYEFAAGLNACLDKVIEFAASKEDLDTLKRLTEEFQAELATLRGRVDSLEARVKELEATRFSTTTKLQGEVIFSLDAVANTAGNERNQDGAVSFGNRVSLNLNTSFTGKDLLLTRLRARNIETIQQRLSPGFNPSGSRLDYDGTGSPGVPNSANTFFLDKLLYRFPVGDVSFTVGTAGVQPQDYGLSDATFFSGPANTKAFKYVGAGVYADTRDADTAGVGFNWKASKNFSFQAGYINRNSADVSTVNSGGVFGFTPTGTGTNSWDVNAQVKYQTDNNKFRVALAYALRN

SEQ ID NO: 11 - protein, 140 aa, Unidentified
  source 1..140: note = synthetic sequence
MTKLKLLALGVLIATSAGVAHAEGKFSLGAGVGVVEHPYKDYDTDVYPVPVINYEGDNFWFRGLGGGYYLWNDATDKLSITAYWSPLYFKAKDSGDHQMRHLDDRKSTMMAGLSYAHFTQYGYLRTTLAGDTLDNSNGIV

SEQ ID NO: 12 - protein, 211 aa, Unidentified
MTLDKALVLRTCANNMADHCGLIWPASGTVESRYWQSTRRHENGLVGLLWGAGTSAFLSVHADARWIVCEVAVADIISLEEPGMVKFPRAEVVHVGDRISASHFISARQADPASTSTSTSTSTLTPMPTAIPTPMPAVASVTLPVAEQARHEVFDVASVSAAAAPVNTLPVTTPQNLQTATYGSTLSGDNHSRLIAGYGSNETAGNHSDLI

SEQ ID NO: 13 - protein, 52 aa, Unidentified
MKRHLNTCYRLVWNHMTGAFVVASELARARGKRGGVAVALSLAAVTSLPVLA

SEQ ID NO: 14 - protein, 27 aa, unidentified
MKRLFSALLLAPAIAGVAAGAANANGL

SEQ ID NO: 15 - protein, 22 aa, unidentified
MKYLLPTAAAGLLLLAAQPAMA

SEQ ID NO: 16 - protein, 53 aa, Unidentified
MNRIYSLRYSAVARGFIAVSEFARKCVHKSVRRLCFPVLLLIPVLFSAGSLAG

SEQ ID NO: 17 - protein, 7 aa, Unidentified
GSSSGSS

SEQ ID NO: 18 - protein, 5 aa, Unidentified
GGGGS

SEQ ID NO: 19 - protein, 23 aa, Unidentified
GGGGSGGSGGGGSGGGGSGGGGS

SEQ ID NO: 20 - protein, 1038 aa, Unidentified
MKRHLNTCYRLVWNHMTGAFVVASELARARGKRGGVAVALSLAAVTSLPVLAADIVVHPGETVNGGTLANHDNQIVFGTTNGMTISTGLEYGPDNEANTGGQWVQDGGTANKTTVTSGGLQRVNPGGSVSDTVISAGGGQSLQGRAVNTTLNGGEQWMHEGAIATGTVINDKGWQVVKPGTVATDTVVNTGAEGGPDAENGDTGQFVRGDAVRTTINKNGRQIVRAEGTANTTVVYAGGDQTVHGHALDTTLNGGYQYVHNGGTASDTVVNSDGWQIVKNGGVAGNTTVNQKGRLQVDAGGTATNVTLKQGGALVTSTAATVTGINRLGAFSVVEGKADNVVLENGGRLDVLTGHTATNTRVDDGGTLDVRNGGTATTVSMGNGGVLLADSGAAVSGTRSDGKAFSIGGGQADALMLEKGSSFTLNAGDTATDTTVNGGLFTARGGTLAGTTTLNNGAILTLSGKTVNNDTLTIREGDALLQGGSLTGNGSVEKSGSGTLTVSNTTLTQKAVNLNEGTLTLNDSTVTTDVIAQRGTALKLTGSTVLNGAIDPTNVTLASGATWNIPDNATVQSVVDDLSHAGQIHFTSTRTGKFVPATLKVKNLNGQNGTISLRVRPDMAQNNADRLVIDGGRATGKTILNLVNAGNSASGLATSGKGIQVVEAINGATTEEGAFVQGNRLQAGAFNYSLNRDSDESWYLRSENAYRAEVPLYASMLTQAMDYDRIVAGSRSHQTGVNGENNSVRLSIQGGHLGHDNNGGIARGATPESSGSYGFVRLEGDLMRTEVAGMSVTAGVYGAAGHSSVDVKDDDGSRAGTVRDDAGSLGGYLNLVHTSSGLWADIVAQGTRHSMKASSDNNDFRARGWGWLGSLETGLPFSITDNLMLEPQLQYTWQGLSLDDGKDNAGYVKFGHGSAQHVRAGFRLGSHNDMTFGEGTSSRAPLRDSAKHSVSELPVNWWVQPSVIRTFSSRGDMRVGTSTAGSGMTFSPSQNGTSLDLQAGLEARVRENITLGVQAGYAHSVSGSSAEGYNGQATLNVT

SEQ ID NO: 21 - protein, 784 aa, Unidentified
MKRHLNTCYRLVWNHMTGAFVVASELARARGKRGGVAVALSLAKKFIGGLVGSLMIGGVALAGGGHVVKWGYVGKIGPAHWGDLAHEYFMCKVGKNQSPVDINSSVTIEAQLEPINFHYRDQISGEIVNNGHTIMVVPKEDNYIVVDGKKFHLKQFHFHSPSEHTVEGKYYLLELHFVHQADDGQLAVIGVVFDRGAEHPEIAKLWKEAPEHEGKKELKSLVNMQALLPENLDYYRYSGSLTTPPCSEGVIWLFLKNPLQISEAQAEKFKKIMGFENNRPVQPVNARKILKGSSSGSSTNVTLASGATWNIPDNATVQSVVDDLSHAGQIHFTSTRTGKFVPATLKVKNLNGQNGTISLRVRPDMAQNNADRLVIDGGRATGKTILNLVNAGNSASGLATSGKGIQVVEAINGATTEEGAFVQGNRLQAGAFNYSLNRDSDESWYLRSENAYRAEVPLYASMLTQAMDYDRIVAGSRSHQTGVNGENNSVRLSIQGGHLGHDNNGGIARGATPESSGSYGFVRLEGDLMRTEVAGMSVTAGVYGAAGHSSVDVKDDDGSRAGTVRDDAGSLGGYLNLVHTSSGLWADIVAQGTRHSMKASSDNNDFRARGWGWLGSLETGLPFSITDNLMLEPQLQYTWQGLSLDDGKDNAGYVKFGHGSAQHVRAGFRLGSHNDMTFGEGTSSRAPLRDSAKHSVSELPVNWWVQPSVIRTFSSRGDMRVGTSTAGSGMTFSPSQNGTSLDLQAGLEARVRENITLGVQAGYAHSVSGSSAEGYNGQATLNVT

SEQ ID NO: 22 - protein, 43 aa, Unidentified
MKRHLNTCYRLVWNHMTGAFVVASELARARGKRGGVAVALSLA

SEQ ID NO: 23 - protein, 487 aa, Unidentified
PTNVTLASGATWNIPDNATVQSVVDDLSHAGQIHFTSTRTGKFVPATLKVKNLNGQNGTISLRVRPDMAQNNADRLVIDGGRATGKTILNLVNAGNSASGLATSGKGIQVVEAINGATTEEGAFVQGNRLQAGAFNYSLNRDSDESWYLRSENAYRAEVPLYASMLTQAMDYDRIVAGSRSHQTGVNGENNSVRLSIQGGHLGHDNNGGIARGATPESSGSYGFVRLEGDLMRTEVAGMSVTAGVYGAAGHSSVDVKDDDGSRAGTVRDDAGSLGGYLNLVHTSSGLWADIVAQGTRHSMKASSDNNDFRARGWGWLGSLETGLPFSITDNLMLEPQLQYTWQGLSLDDGKDNAGYVKFGHGSAQHVRAGFRLGSHNDMTFGEGTSSRAPLRDSAKHSVSELPVNWWVQPSVIRTFSSRGDMRVGTSTAGSGMTFSPSQNGTSLDLQAGLEARVRENITLGVQAGYAHSVSGSSAEGYNGQATLNVT

SEQ ID NO: 24 - DNA, 6795 bp, Unidentified
  source 1..6795: mol_type = other DNA
ctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaagatcgataatctcatgaccaaaatcccttaacgtgagttttcgttccactgagcgtcagaccccgtataaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgtgtgctgggccccaatgccttctccaagggcggcattcccctgactgttgaaggcgttgccaatatcaagattgctggggaagaaccgaccatccacaacgcgatcgagcggctgcttggcaaaaaccgtaaggaaatcgagcaaattgccaaggagaccctcgaaggcaacttgcgtggtgttttagccagcctcacgccggagcagatcaacgaggacaaaattgcctttgccaaaagtctgctggaagaggcggaggatgaccttgagcagctgggtcaagtcctcgatacgctgcaagtccagaacatttccgatgaggtcggttatctctcggctagtggacgcaagcagcgggctgatctgcagcgagatgcccgaattgctgaagccgatgcccaggctgcctctgcgatccaaacggccgaaaatgacaagatcacggccctgcgtcggatcgatcgcgatgtagcgatcgcccaagccgaggccgagcgccggattcaggatgcgttgacgcggcgcgaagcggtggtggccgaagctgaagcggacattgctaccgaagtcgctcgtagccaagcagaactccctgtgcagcaggagcggatcaaacaggtgcagcagcaacttcaagccgatgtgatcgccccagctgaggcagcttgtaaacgggcgatcgcggaagcgcggggggccgccgcccgtatcgtcgaagatggaaaagctcaagcggaagggacccaacggctggcggaggcttggcagaccgctggtgctaatgcccgcgacatcttcctgctccagaagtctagataatccctagcgatcgcaagtccaaaggttgtctacaatcaatatccaagcatcaaaaagcgccccattcgaggcgctttttgattattcagactgctgtaattccggcaattaggttatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagataccagcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcataatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgccgttaccaccgctgcgttcggtcaaggttctggaccagttgcgtgagcgcatacgctacttgcattacagcttacgaaccgaacaggcttatgtccactgggttcgtgccttcatccgtttccacggtgtgcgtcacccggcaaccttgggtagcagcgaagtcgaggcatttctgtcctggctggctatttagcgtcttctaatccagtgtagacagtagttttggctccgttgagcactgtagccttgggcgatcgctctaaacattacataaattcacaaagttttcgttacataaaaatagtgtctacttagctaaaaattaagggttttttacacctttttgacagttaatctcctagcctaaaaagcaagagtttttaactaagactcttgccctttacaacctcgaaggagcgtcagatctcatatgaaacgccacctgaacacctgctaccgcctggtgtggaaccacatgaccggcgcctttgtggtggccagcgaactggcccgcgcccgcggcaaacgcggcggcgtggccgtggccctgagcctggccgccgtgaccagcctgcccgtgctggccggcggcggccacgtggtgaaatggggctacgtgggcaaaatcggccccgcccactggggcgatctggcccacgaatactttatgtgcaaagtgggcaaaaaccagagccccgtggatatcaacagcagcgtgaccatcgaagcccagctggaacccatcaactttcactaccgcgatcagatcagcggcgaaatcgtgaacaacggccacaccatcatggtggtgcccaaagaagataactacatcgtggtggatggcaaaaaatttcacctgaaacagtttcactttcacagccccagcgaacacaccgtggaaggcaaatactacctgctggaactgcactttgtgcaccaggccgatgatggccagctggccgtgatcggcgtggtgtttgatcgcggcgccgaacaccccgaaatcgccaaactgtggaaagaagcccccgaacacgaaggcaaaaaagaactgaaaagcctggtgaacatgcaggccctgctgcccgaaaacctggattactaccgctacagcggcagcctgaccacccccccctgcagcgaaggcgtgatctggctgtttctgaaaaaccccctgcagatcagcgaagcccaggccgaaaaatttaaaaaaatcatgggctttgaaaacaaccgccccgtgcagcccgtgaacgcccgcaaaatcctgaaaggcagcagcagcggcagcagccccaccaacgtgaccctggccagcggcgccacctggaacatccccgataacgccaccgtgcagagcgtggtggatgatctgagccacgccggccagatccactttaccagcacccgcaccggcaaatttgtgcccgccaccctgaaagtgaaaaacctgaacggccagaacggcaccatcagcctgcgcgtgcgccccgatatggcccagaacaacgccgatcgcctggtgatcgatggcggccgcgccaccggcaaaaccatcctgaacctggtgaacgccggcaacagcgccagcggcctggccaccagcggcaaaggcatccaggtggtggaagccatcaacggcgccaccaccgaagaaggcgcctttgtgcagggcaaccgcctgcaggccggcgcctttaactacagcctgaaccgcgatagcgatgaaagctggtacctgcgcagcgaaaacgcctaccgcgccgaagtgcccctgtacgccagcatgctgacccaggccatggattacgatcgcatcgtggccggcagccgcagccaccagaccggcgtgaacggcgaaaacaacagcgtgcgcctgagcatccagggcggccacctgggccacgataacaacggcggcatcgcccgcggcgccacccccgaaagcagcggcagctacggctttgtgcgcctggaaggcgatctgatgcgcaccgaagtggccggcatgagcgtgaccgccggcgtgtacggcgccgccggccacagcagcgtggatgtgaaagatgatgatggcagccgcgccggcaccgtgcgcgatgatgccggcagcctgggcggctacctgaacctggtgcacaccagcagcggcctgtgggccgatatcgtggcccagggcacccgccacagcatgaaagccagcagcgataacaacgattttcgcgcccgcggctggggctggctgggcagcctggaaaccggcctgccctttagcatcaccgataacctgatgctggaaccccagctgcagtacacctggcagggcctgagcctggatgatggcaaagataacgccggctacgtgaaatttggccacggcagcgcccagcacgtgcgcgccggctttcgcctgggcagccacaacgatatgacctttggcgaaggcaccagcagccgcgcccccctgcgcgatagcgccaaacacagcgtgagcgaactgcccgtgaactggtgggtgcagcccagcgtgatccgcacctttagcagccgcggcgatatgcgcgtgggcaccagcaccgccggcagcggcatgacctttagccccagccagaacggcaccagcctggatctgcaggccggcctggaagcccgcgtgcgcgaaaacatcaccctgggcgtgcaggccggctacgcccacagcgtgagcggcagcagcgccgaaggctacaacggccaggccaccctgaacgtgaccttttaacatatgcaccaccaccatcaccacgaaaacctgtactttcagggcaagcttcgaattcccggatccgcggtaccaggcaaacccatccccaaccccctgctgggcctggatagcaccggtggtggtcaccaccaccatcaccactagagtactgtatgcatcgagtgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggccttctcgagtccctgctcgtcacgctttcaggcaccgtgccagatatcgacgtggagtcgatcactgtgattggcgaaggggaaggcagcgctacccaaatcgctagcttgctggagaagctgaaacaaaccacgggcattgatctggcgaaatccctaccgggtcaatccgactcgcccgctgcgaagtcctaagagatagcgatgtgaccgcgatcgcttgtcaagaatcccagtgatcccgaaccataggaaggcaagctcaatgcttgcctcgtcttgaggactatctagatgtctgtggaacgcacatttattgccatcaagcccgatggcgttcagcggggtttggtcggtacgatcatcggccgctttgagcaaaaaggcttcaaactggtgggcctaaagcagctgaagcccagtcgcgagctggccgaacagcactatgctgtccaccgcgagcgccccttcttcaatggcctcgtcgagttcatcacctctgggccgatcgtggcgatcgtcttggaaggcgaaggcgttgtggcggctgctcgcaagttgatcggcgctaccaatccgctgacggcagaaccgggcaccatccgtggtgattttggtgtcaatattggccgcaacatcatccatggctcggatgcaatcgaaacagcacaacaggaaattgctctctggtttagcccagcagagctaagtgattggacccccacgattcaaccctggctgtacgaataaggtctgcattccttcagagagacattgccatgccg

SEQ ID NO: 25 - protein, 23 aa, Unidentified
GGGGSGGSGGGGSGGGGSGGGGS

(19) United States
(12) Patent Application Publication
(10) Pub. No.: US 2025/0367648 A1
(43) Pub. Date: Dec. 4, 2025

(54) ENZYME-DECORATED NANOCATALYSTS

(71) Applicant: Cassiopea LLC, Phoenix, AZ (US)

(72) Inventor: Keith LADEROUTE, Redwood City, CA (US)

(21) Appl. No.: 19/222,494

(22) Filed: May 29, 2025

Related U.S. Application Data
(60) Provisional application No. 63/653,187, filed on May 29, 2024.

(51) Int. Cl. B01J 31/00; B01J 35/40; C07K 14/00; C12N 1/12; C12N 1/20; C12N 9/88; C12P 3/00; C12R 1/89

(52) U.S. Cl. CPC B01J 31/003; B01J 35/40; C07K 14/00; C12N 1/12; C12N 1/20; C12N 9/88; C12P 3/00; C12Y 402/01001; C07K 2319/00; C12R 2001/89

(57) ABSTRACT

Provided herein are vesicular nanocatalysts decorated on their surface with enzymes, such as for industrial use. More particularly, the nanocatalysts include outer membrane vesicles (OMVs) presenting on their surface carbonic anhydrase or a functional fragment or derivative thereof. Uses, methods of manufacture, and compositions of the nanocatalysts are also disclosed herein.

### 1. SEQUENCE LISTING

The instant application contains a Sequence Listing which has been submitted via Patent Center and is hereby incorporated by reference in its entirety. Said XML copy, created on May 28, 2025, is named 42201-58430WO_SEQLISTING, and is 40,483 bytes in size.

### 2. FIELD

The present disclosure generally relates to enzyme-decorated vesicular nanocatalysts, such as for industrial use.

### 3. BACKGROUND

The outer membrane vesicles (OMVs) derived from Gram-negative bacteria have been acknowledged for their biomedical applications, including drug delivery, vaccine development, and bioengineering. They are spherical structures naturally secreted by these bacteria, encompassing an assortment of substances such as proteins, nucleic acids, and lipopolysaccharides—each varying based on the type and state of the parent bacterium. In recent years, technologies have emerged enabling the horizontal gene transfer and genetic manipulation of bacteria to generate bespoke OMVs with desired attributes.

Despite these advances, the engineered OMVs remains vastly underutilized. It is due to several problems that still need to be addressed—for example, the assembly mechanism of OMVs hasn't been fully understood and the separation and purification processes of OMVs are complex and require further studies. In large-scale production, the size of OMVs is not easy to control, and the difference between batches is large. The lack of understanding of the exact assembly mechanism of OMVs results in difficulties in engineering the particles.

### 4. SUMMARY

The present disclosure concerns outer membrane vesicle (OMV) nanocatalysts presenting on their surface an enzyme. In particular embodiments, the OMV nanocatalysts present carbonic anhydrase or a functional fragment or derivative thereof. The present disclosure further relates to uses of the nanocatalysts, such as for environmental remediation, food technology, and specialty and commodity organic and inorganic chemical synthesis; methods of manufacturing the nanocatalysts; and compositions comprising the nanocatalysts.

In some embodiments, provided herein is an engineered outer membrane vesicle (OMV) comprising a heterologous enzyme presented on the outer surface of the engineered OMV. In some embodiments, the heterologous enzyme is operably linked to a transmembrane protein embedded in the membrane of the engineered OMV to form a fusion protein. In some embodiments, the transmembrane protein is derived from an autotransporter protein (ATP) or an outer membrane protein (OMP).

In some embodiments, according to any of the engineered OMVs described herein, the transmembrane protein is derived from an ATP selected from antigen 43 (Ag43), hemoglobin-binding protease (Hbp), pertactin, extracellular serine protease plasmid-encoded (EspP), IgA1 protease, esterase autotransporter (EstA), adhesion and penetration protein (Hap), adhesin involved in diffuse adherence (AIDA-I), plasmid-encoded toxin (Pet), protease involved in intestinal colonization (Pic), temperature-sensitive hemagglutinin (Tsh), shigella extracellular protein (SepA), and vacuolating cytotoxin A (VacA). In some embodiments, the ATP is Ag43. In some embodiments, the transmembrane protein comprises the amino acid sequence of SEQ ID NO: 8. In some embodiments, the ATP is Hbp. In some embodiments, the transmembrane protein comprises the amino acid sequence of SEQ ID NO: 9.

In some embodiments, according to any of the engineered OMVs described herein, the transmembrane protein is derived from an OMP selected from Synechococcus outer membrane protein A (SomA), MipA, and ice nucleation proteins (INPs). In some embodiments, the OMP is SomA. In some embodiments, the transmembrane protein comprises the amino acid sequence of SEQ ID NO: 10. In some embodiments, the OMP is MipA. In some embodiments, the transmembrane protein comprises the amino acid sequence of SEQ ID NO: 11. In some embodiments, the OMP is an INP. In some embodiments, the INP is from Pseudomonas syringae. In some embodiments, the transmembrane protein comprises the amino acid sequence of SEQ ID NO: 12.

In some embodiments, according to any of the engineered OMVs described herein, the fusion protein further comprises a linker connecting the heterologous enzyme to the transmembrane protein. In some embodiments, the linker is a peptide linker comprising the amino acid sequence of any one of SEQ ID NOs: 17-19 and 25.

In some embodiments, according to any of the engineered OMVs described herein, the engineered OMV comprises at least about 10 heterologous enzyme molecules presented on its outer surface.

In some embodiments, according to any of the engineered OMVs described herein, the engineered OMV has a diameter of about 50 nm to about 250 nm. In some embodiments, the engineered OMV has a diameter of about 70 nm to about 130 nm.

In some embodiments, according to any of the engineered OMVs described herein, the engineered OMV is obtained from a photosynthetic microorganism. In some embodiments, according to any of the engineered OMVs described herein, the engineered OMV is expressed by a photosynthetic microorganism. In some embodiments, the photosynthetic organism is an engineered photosynthetic organism.

In some embodiments, the photosynthetic microorganism is a gram-negative bacterium. In some embodiments, the gram-negative bacterium is a cyanobacterium. In some embodiments, the cyanobacterium is selected from Synechocystis sp., Synechococcus sp., Microcystis aeruginosa, Leptolyngbya boryana, Cyanobium gracile, Phormidium persicinum, and Gloeocapsa sp. In some embodiments, the cyanobacterium is selected from Synechococcus sp. PCC 7942, Synechococcus elongatus UTEX 2973, and Synechocystis sp. PCC 6803. In some embodiments, the photosynthetic microorganism is a eukaryotic microalgae.

In some embodiments, according to any of the engineered OMVs described herein, the heterologous enzyme comprises a carbonic anhydrase (CA) polypeptide or functional fragment thereof. In some embodiments, the CA polypeptide is derived from a thermostable CA. In some embodiments, the thermostable CA is from Thermosulfurimonas dismutans. In some embodiments, the CA polypeptide comprises the amino acid sequence of SEQ ID NO: 7.

In some embodiments, provided herein is an engineered host cell capable of producing an OMV comprising a heterologous enzyme presented on the outer surface of the OMV, wherein the engineered host cell is a photosynthetic microorganism comprising heterologous nucleic acid encoding a fusion protein comprising the heterologous enzyme operably linked to a transmembrane protein, and wherein the fusion protein is capable of being inserted into the outer membrane of the engineered host cell such that the heterologous enzyme is presented on the outer surface of the engineered host cell.

In some embodiments, according to any of the engineered host cells described herein, the transmembrane protein is derived from an autotransporter protein (ATP) or an outer membrane protein (OMP). In some embodiments, the transmembrane protein is derived from an ATP selected from antigen 43 (Ag43), hemoglobin-binding protease (Hbp), pertactin, extracellular serine protease plasmid-encoded (EspP), IgA1 protease, esterase autotransporter (EstA), adhesion and penetration protein (Hap), adhesin involved in diffuse adherence (AIDA-I), plasmid-encoded toxin (Pet), protease involved in intestinal colonization (Pic), temperature-sensitive hemagglutinin (Tsh), shigella extracellular protein (SepA), and vacuolating cytotoxin A (VacA). In some embodiments, the ATP is Ag43. In some embodiments, the transmembrane protein comprises the amino acid sequence of SEQ ID NO: 8. In some embodiments, the ATP is Hbp. In some embodiments, the transmembrane protein comprises the amino acid sequence of SEQ ID NO: 9.

In some embodiments, according to any of the engineered host cells described herein, the transmembrane protein is derived from an OMP selected from Synechococcus outer membrane protein A (SomA), MipA, and ice nucleation proteins (INPs). In some embodiments, the OMP is SomA. In some embodiments, the transmembrane protein comprises the amino acid sequence of SEQ ID NO: 10. In some embodiments, the OMP is MipA. In some embodiments, the transmembrane protein comprises the amino acid sequence of SEQ ID NO: 11. In some embodiments, the OMP is an INP. In some embodiments, the INP is from Pseudomonas syringae. In some embodiments, the transmembrane protein comprises the amino acid sequence of SEQ ID NO: 12.

In some embodiments, according to any of the engineered host cells described herein, the fusion protein further comprises a linker connecting the heterologous enzyme to the transmembrane protein. In some embodiments, the linker is a peptide linker comprising the amino acid sequence of any one of SEQ ID NOs: 17-19 and 25. In some embodiments, the linker comprises an amino acid sequence of GGG.

In some embodiments, according to any of the engineered host cells described herein, the fusion protein further comprises a signal peptide capable of targeting the fusion protein to the inner membrane of the engineered host cell. In some embodiments, the signal peptide comprises the amino acid sequence of any one of SEQ ID NOs: 13-16.

In some embodiments, according to any of the engineered host cells described herein, the engineered host cell is a cyanobacterium. In some embodiments, the cyanobacterium is selected from Synechocystis sp., Synechococcus sp., Microcystis aeruginosa, Leptolyngbya boryana, Cyanobium gracile, Phormidium persicinum, and Gloeocapsa sp. In some embodiments, the cyanobacterium is selected from Synechococcus sp. PCC 7942, Synechococcus elongatus UTEX 2973, and Synechocystis sp. PCC 6803.

In some embodiments, according to any of the engineered host cells described herein, the engineered host cell is a eukaryotic microalgae.

In some embodiments, according to any of the engineered host cells described herein, the heterologous enzyme comprises a CA polypeptide or functional fragment thereof. In some embodiments, the CA polypeptide is derived from a thermostable CA. In some embodiments, the thermostable CA is from Thermosulfurimonas dismutans. In some embodiments, the CA polypeptide comprises the amino acid sequence of SEQ ID NO: 7.

In some embodiments, according to any of the engineered host cells described herein, the heterologous nucleic acid is present extrachromosomally. In some embodiments, the heterologous nucleic acid is a plasmid.

In some embodiments, according to any of the engineered host cells described herein, the heterologous nucleic acid is integrated into the chromosome of the engineered host cell. In some embodiments, the heterologous nucleic acid is integrated into the chromosome by site-specific targeting. In some embodiments, the heterologous nucleic acid is integrated into the chromosome by site-specific targeting using a CRISPR/Cas9 system.

In some embodiments, according to any of the engineered host cells described herein, the heterologous nucleic acid is operably linked to an inducible promoter.

In some embodiments, provided herein is a method of producing an engineered OMV comprising: (a) culturing an engineered host cell according to any of the embodiments described herein under conditions suitable for expression of the fusion protein; and (b) obtaining an OMV produced by the engineered host cell, thereby producing the engineered OMV. In some embodiments, the engineered host cell is cultured in the presence of CO2 and light. In some embodiments, the method further comprises filtering the engineered OMV. In some embodiments, the filtering is performed by ultrafiltration. In some embodiments, the filtering is performed by a membrane bioreactor. In some embodiments, the method further comprises concentrating the engineered OMV.

In some embodiments, provided herein is a nanocatalyst composition comprising an engineered OMV according to any of the embodiments described herein. In some embodiments, the engineered OMV is produced by a method of producing an engineered OMV according to any of the embodiments described herein.

In some embodiments, according to any of the nanocatalyst compositions described herein, the composition comprises the engineered OMV at a concentration greater than about 100 mg/L.

In some embodiments, according to any of the nanocatalyst compositions described herein, the composition comprises the engineered OMV in an amount greater than about 10 g.

In some embodiments, according to any of the nanocatalyst compositions described herein, the composition is not a therapeutic composition.

In some embodiments, provided herein is a nanocatalyst product comprising a nanocatalyst composition according to any of the embodiments described herein in a volume greater than about 1L

In some embodiments, provided herein is a method of reducing CO₂ comprising interacting an engineered OMV according to any of the embodiments described herein wherein the heterologous enzyme comprises a carbonic anhydrase (CA) polypeptide or functional fragment thereof with CO₂ under a condition suitable for the CA polypeptide or functional fragment thereof to convert CO₂ to a catalysis product. In some embodiments, the catalysis product is a metal carbonate. In some embodiments, the engineered OMV is interacted with CO₂ in the presence of H₂O and a metal ion under alkaline conditions suitable for (i) the conversion of CO₂ to CO₃²⁻ by the CA polypeptide or functional fragment thereof, and (ii) association of CO₃²⁻ with the metal ion to form the metal carbonate. In some embodiments, the metal ion is Ca²⁺, Mg²⁺, Fe²⁺, Zn²⁺, Cu²⁺, Mn²⁺, or Li⁺.

In some embodiments, according to any of the methods of reducing CO2 described herein, the engineered OMV is interacted with CO2 in air, water, or waste.

In some embodiments, according to any of the methods of reducing CO2 described herein, the method further comprises producing an engineered OMV according to any of the methods described herein. In some embodiments, producing the engineered OMV is carried out in a first reactor and producing the catalysis product is carried out in a second reactor. In some embodiments, the method further comprises isolating engineered OMVs produced in the first reactor and introducing the isolated engineered OMVs into the second reactor. In some embodiments, isolating the engineered OMVs is carried out by filtration.

In some embodiments, provided herein is a system for the production of an engineered OMV according to any of the embodiments described herein, the system comprising: (a) a first reactor configured to receive a microorganism capable of producing the engineered OMV: (b) a receptacle configured to receive the engineered OMV produced in the first reactor and (c) a filtration interface connecting the first reactor to the receptable, wherein the engineered OMV produced in the first reactor is capable of passing through the filtration interface into the receptacle.

In some embodiments, according to any of the systems for the production of an engineered OMV described herein, the first reactor is a photobioreactor configured to receive light, and the microorganism is a photosynthetic microorganism. In some embodiments, the photosynthetic microorganism is a cyanobacterium or a eukaryotic microalga. In some embodiments, the photosynthetic microorganism is an engineered host cell according to any of the embodiments described herein.

In some embodiments, according to any of the systems for the production of an engineered OMV described herein, the first reactor is configured to receive ambient air/CO₂.

In some embodiments, according to any of the systems for the production of an engineered OMV described herein, the microorganism in the first reactor is incapable or substantially incapable of passing through the filtration interface into the receptacle.

In some embodiments, according to any of the systems for the production of an engineered OMV described herein, the filtration interface comprises one or more filtration membranes. In some embodiments, the filtration interface comprises at least 2, 3, 4, or 5 filtration membranes. In some embodiments, the one or more filtration membranes are one or more ultrafiltration membranes.

In some embodiments, according to any of the systems for the production of an engineered OMV described herein, the receptacle is configured to receive the engineered OMV: (a) at a concentration greater than about 100 mg/L; (b) in an amount greater than about 10 g; and/or (c) in a volume greater than about 100 mL.

In some embodiments, provided herein is a system for catalyzing the conversion of CO2 and H2O to HCO3− and H+, the system comprising: (a) a first reactor configured to receive a microorganism capable of producing an engineered OMV according to any of the embodiments described herein: (b) a second reactor configured to receive the engineered OMV produced in the first reactor in conditions suitable for carrying out the conversion of CO2 and H2O to HCO3− and H+; and (c) a filtration interface connecting the first reactor to the second reactor, wherein the engineered OMV produced in the first reactor is capable of passing through the filtration interface into the second reactor.

In some embodiments, according to any of the systems for catalyzing the conversion of CO₂ and H₂O to HCO₃⁻ and H⁺ described herein, the first reactor is a photobioreactor configured to receive light, and the microorganism is a photosynthetic microorganism. In some embodiments, the photosynthetic microorganism is a cyanobacterium or a eukaryotic microalga. In some embodiments, the photosynthetic microorganism is an engineered host cell according to any of the embodiments described herein.

In some embodiments, according to any of the systems for catalyzing the conversion of CO₂ and H₂O to HCO₃⁻ and H⁺ described herein, the first reactor is configured to receive ambient air/CO₂.

In some embodiments, according to any of the systems for catalyzing the conversion of CO₂ and H₂O to HCO₃⁻ and H⁺ described herein, the second reactor is an alkaline reactor comprising an aqueous composition having a pH greater than about 8.0.

In some embodiments, according to any of the systems for catalyzing the conversion of CO₂ and H₂O to HCO₃⁻ and H⁺ described herein, the second reactor is configured to receive CO₂.

In some embodiments, according to any of the systems for catalyzing the conversion of CO₂ and H₂O to HCO₃⁻ and H⁺ described herein, the second reactor comprises an aqueous composition comprising a metal ion under conditions suitable for the formation of a metal carbonate comprising the metal ion. In some embodiments, the metal ion is Ca²⁺, Mg²⁺, Fe²⁺, Zn²⁺, Cu²⁺, Mn²⁺, or Li⁺.

In some embodiments, according to any of the systems for catalyzing the conversion of CO₂ and H₂O to HCO₃⁻ and H⁺ described herein, the microorganism in the first reactor is incapable or substantially incapable of passing through the filtration interface into the second reactor.

In some embodiments, according to any of the systems for catalyzing the conversion of CO₂ and H₂O to HCO₃⁻ and H⁺ described herein, the filtration interface comprises one or more filtration membranes. In some embodiments, the filtration interface comprises at least 2, 3, 4, or 5 filtration membranes. In some embodiments, the one or more filtration membranes are one or more ultrafiltration membranes.

In some embodiments, according to any of the systems for catalyzing the conversion of CO₂ and H₂O to HCO₃⁻ and H⁺ described herein, the second reactor is configure to receive the engineered OMV: (a) at a concentration greater than about 100 mg/L; (b) in an amount greater than about 10 g; and/or (c) in a volume greater than about 100 mL.

### 5. BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 provides graphical depiction of an OMV nanocatalyst in cross-section and as a sphere showing surface decoration with carbonic anhydrase/CA enzyme (OMV-CA nanocatalysts).

FIG. 2 provides schematic diagram of an exemplary platform for the synthesis, isolation, and function of OMV-CA (carbonic anhydrase) nanocatalysts for carbon capture, storage, and utilization.

FIG. 3 illustrates a method for scaling up the OMV-CA (carbonic anhydrase) nanocatalyst platform for industrial application.

FIG. 4 illustrates the pathway by which an autotransporter such as Ag43 transports a protein domain to the surface of the OM of a gram-negative bacterial cell. Bottom: schematic diagram of a recombinant fusion protein design based on the Ag43 autotransporter, including a signal peptide (SP) for transport to the periplasm, an a domain (passenger), and a β domain (translocation unit).

FIG. 5 provides an anti-TdCA immunoblot of whole cell lysates of UTEX 2973 cells transformed with a TdCA-Ag43 construct (78 kDa). LDR/Ladder indicates sized protein standards, the sizes of which are indicated in the margin. WT indicates the wild type UTEX 2973 strain, whereas A, B and D are separate cultures originating from distinct colonies formed following antibiotic selection. Expected sizes of different fusion proteins are (i) TdCA-Ag43 with SP (signal peptide): 84 kDa: (ii) TdCA-Ag43 without SP (signal peptide): 80 kDa: (iii) TdCA: 28 kDa; and (iv) Ag43: 52 kDa.

FIG. 6 provides carbonic anhydrase activity assay results of washed whole cyanobacterial cells, corrected for the optical density of the 10× concentrated culture. WT indicates wild type cells, and other samples are distinct transformed cultures (TdCA-Ag43) representing different colonies from an antibiotic selection agar plate.

FIG. 7 provides TdCA-Ag43 immunoblotting results for separate treatments of whole cell samples of clonal culture D with proteinase K at different concentrations. The band near 78 kDa, which corresponds to the expected size of the TdCA-Ag43 fusion protein, shows significant decrease in intensity with increasing proteinase K concentrations.

FIG. 8 provides quantitative analysis (measuring band volume/density) of TdCA-Ag43 immunoblotting results for sample D (Units proteinase K/mL) in FIG. 8.

FIG. 9 shows the carbonic anhydrase activity of OMVs from the UTEX 2973 cell supernatants.

### 6. DETAILED DESCRIPTION OF THE INVENTION

### A. Definitions

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

As used herein, the singular terms “a,” “an,” and “the” include the plural reference unless the context clearly indicates otherwise. Thus, for example, reference to “a peptide sequence” includes a plurality of such sequences and so forth.

An “autotransporter” is a protein that belongs to the pfam autotransporter family (‘Autotransporter’ PF03797) and that also is known or predicted to form a beta stem motif. The BETAWRAPPRO method for sequence analysis can be used to predict if the passenger domain of an autotransporter will form a beta stem motif (Junker et al 2006 Proc Natl Acad Sci USA 103 (13): 4918-23).

“Beta stem forming sequence” refers to the sequence of a passenger domain of an autotransporter that forms a beta stem structure. The beta stem forming sequence of a passenger can be identified using crystal structure determination. As described above the beta stem forming sequence may alternatively be identified using the M4T homology modeling method (Rykunov et al 2009 J Struct Funct Genomics 10:95-99) or similar prediction methods.

A “side domain” is a domain that is part of the passenger domain but is not part of the beta stem. Typically, a side domain is located in the passenger domain between two stretches of beta stem forming sequence. A side domain starts at the first amino acid after the preceding beta strand and it ends one amino acid before the starting amino acid of the beta strand following the side domain. The side domain can also be located at the N-terminus of the passenger domain. Autotransporters may have several side domains.

A moiety is “displayed” on an object, such as a cell or vesicle, or the object is “decorated” with the moiety, when it remains associated with the outer membrane of the host cell such that it at least partly protrudes outside the cell. The secreted protein may be attached to the cell membrane or a component that resides therein (such as the translocator domain from an autotransporter) in a covalent or non-covalent manner.

Unless otherwise indicated, the terms “oligonucleotides” and “nucleic acids” are used interchangeably and are written left to right in 5′ to 3′ orientation: amino acid sequences are written left to right in amino to carboxy orientation, respectively.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or substantial substitution), insertion of one or more (e.g., generally fewer than 5, 4, or 3) amino acids adjacent to said residue/position, and/or deletion of said residue/position.

The term “sequence identity” refers to a relationship between the sequences of two or more biological molecules (e.g., a pair of polynucleotides or multiple polypeptides), as determined by aligning and comparing the respective sequences. “Percent (%) amino acid sequence identity” with respect to a reference amino acid sequence (e.g., a reference polypeptide) is defined as the percentage of amino acid residues in a candidate sequence that are identical with the amino acid residues in the reference amino acid sequence, after aligning the two sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

The term “amino acid” refers to naturally occurring and non-naturally occurring alpha-amino acids, as well as alpha-amino acid analogs and amino acid mimetics that function in a manner similar to the naturally occurring alpha-amino acids. Amino acids may be referred to herein by either their commonly known three letter symbols or by the one-letter symbols recommended by the IUPAC-IUB Biochemical Nomenclature Commission.

The term “variant” when used in relation to a peptide or polypeptide may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of a polypeptide may result from one or more (such as, for example, about 1 to about 25, about 1 to about 20, about 1 to about 15, about 1 to about 10, or about 1 to about 5) changes to an amino acid sequence of a native or previously unmodified polypeptide. Polypeptide variants may be prepared from the corresponding nucleic acid molecules encoding the variants. In specific embodiments, a variant of a polypeptide retains at least one functional activity of the polypeptide.

A “functional fragment” of a polypeptide will exhibit at least one if not some or all of the biological functions attributed to the intact polypeptide.

The term “fusion,” “fuse” or other grammatical variants thereof when used in relation to a peptide or polypeptide refers to the joining of a peptide or polypeptide, or fragment, variant, and/or derivative thereof, with a heterologous peptide or polypeptide.

The term “attenuation” or “attenuate,” when used herein, refers to partial (such as, 1%, 2%, 5%, 10%, 20%, 25%, 50%, 75%, 90%, 95%, 99%) and less than complete (i.e., 100%) reduction of a given activity.

The term “inhibition,” “inhibit,” or “abolish” when used herein, refers to a complete (i.e., 100%) reduction of a given activity. In some embodiments, a complete reduction is manifested as no or insignificantly minimal or ignorable detection of the activity using a method (e.g., assay) that has been established and validated for detecting such activity.

The term “vector” refers to a substance that is used to carry or include a nucleic acid sequence, including for example, a nucleic acid sequence encoding a polypeptide as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed, both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product, and it is further understood that expression levels can be optimized to obtain sufficient expression using methods well known in the art.

### B. Compositions and Methods of Making the Same

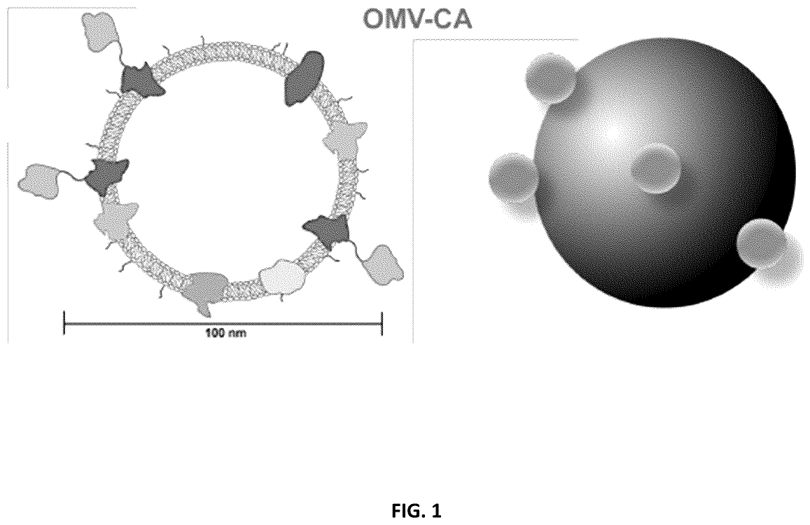

The present disclosure provides novel enzyme-decorated OMVs, i.e., OMVs (FIG. 1) engineered to present a heterologous enzyme on the outer surface. Enzymes contemplated for surface presentation include those having a carbonic anhydrase activity, but not limited thereto. As illustrated in further detail herein, enzyme-decorated OMVs can be designed and configured in multiple ways to achieve specific goals.

In one aspect of the present disclosure, provided herein is an engineered OMV comprising a heterologous enzyme presented on the outer surface of the engineered OMV. In some embodiments, the heterologous enzyme is operably linked to a transmembrane protein embedded in the membrane of the engineered OMV to form a fusion protein. In some embodiments, the transmembrane protein is derived from an autotransporter protein (ATP) or an outer membrane protein (OMP).

Thus, in some embodiments, provided herein is an engineered OMV comprising a heterologous enzyme presented on the outer surface of the engineered OMV, wherein the heterologous enzyme is operably linked to a transmembrane protein embedded in the membrane of the engineered OMV to form a fusion protein, and wherein the transmembrane protein is derived from an ATP. In some embodiments, the ATP is selected from antigen 43 (Ag43), hemoglobin-binding protease (Hbp), pertactin, extracellular serine protease plasmid-encoded (EspP), IgA1 protease, esterase autotransporter (EstA), adhesion and penetration protein (Hap), adhesin involved in diffuse adherence (AIDA-I), plasmid-encoded toxin (Pet), protease involved in intestinal colonization (Pic), temperature-sensitive hemagglutinin (Tsh), shigella extracellular protein (SepA), and vacuolating cytotoxin A (VacA). In some specific embodiments, the ATP is Ag43. In some such embodiments, the transmembrane protein comprises the amino acid sequence of SEQ ID NO: 8, or a variant thereof having at least 90% sequence identity thereto. In some such embodiments, the transmembrane protein comprises an amino acid sequence having at least 80%, at least 85%, at least 90%, at least 95%, at least 97%, or at least 99% sequence identity to SEQ ID NO: 8. In yet other specific embodiments, the ATP is Hbp. In some such embodiments, the transmembrane protein comprises the amino acid sequence of SEQ ID NO: 9, or a variant thereof having at least 90% sequence identity thereto. In some such embodiments, the transmembrane protein comprises an amino acid sequence having at least 80%, at least 85%, at least 90%, at least 95%, at least 97%, or at least 99% sequence identity to SEQ ID NO: 9. In some such embodiments, the transmembrane protein comprises the amino acid sequence of SEQ ID NO: 23, or a variant thereof having at least 90% sequence identity thereto. In some such embodiments, the transmembrane protein comprises an amino acid sequence having at least 80%, at least 85%, at least 90%, at least 95%, at least 97%, or at least 99% sequence identity to SEQ ID NO: 23.

In some other embodiments, provided herein is an engineered outer membrane vesicle (OMV) comprising a heterologous enzyme presented on the outer surface of the engineered OMV, wherein the heterologous enzyme is operably linked to a transmembrane protein embedded in the membrane of the engineered OMV to form a fusion protein, and wherein the transmembrane protein is derived from an OMP selected from Synechococcus outer membrane protein A (SomA), MipA, and ice nucleation proteins (INPs). In some specific embodiments, the OMP is SomA. In some such embodiments, the transmembrane protein comprises the amino acid sequence of SEQ ID NO: 10, or a variant thereof having at least 90% sequence identity thereto. In some such embodiments, the transmembrane protein comprises an amino acid sequence having at least 80%, at least 85%, at least 90%, at least 95%, at least 97%, or at least 99% sequence identity to SEQ ID NO: 10. In yet other specific embodiments, the OMP is MipA. In some such embodiments, the transmembrane protein comprises the amino acid sequence of SEQ ID NO: 11, or a variant thereof having at least 90% sequence identity thereto. In some such embodiments, the transmembrane protein comprises an amino acid sequence having at least 80%, at least 85%, at least 90%, at least 95%, at least 97%, or at least 99% sequence identity to SEQ ID NO: 11. In yet other specific embodiments, the OMP is an INP. In some such embodiments, the INP is from Pseudomonas syringae. In some embodiments, the transmembrane protein comprises the amino acid sequence of SEQ ID NO: 12, or a variant thereof having at least 90% sequence identity thereto. In some such embodiments, the transmembrane protein comprises an amino acid sequence having at least 80%, at least 85%, at least 90%, at least 95%, at least 97%, or at least 99% sequence identity to SEQ ID NO: 12.

In some embodiments, according to any of the engineered OMVs described herein comprising a fusion protein, the fusion protein further comprises a linker connecting the heterologous enzyme to the transmembrane protein. In some embodiments, the linker is a peptide linker comprising the amino acid sequence of any one of SEQ ID NOs: 17-19 and 25.

In some embodiments, according to any of the engineered OMVs described herein, the engineered OMV comprises at least about 10 (including, for example, at least about any of 20, 30, 40, 50, 60, 70, 80, 90, 100, 200, 300, 400, 500, 600, 700, 800, 900, 1000, 2000, 3000, 4000, 5000, or more, including any ranges between any of these values) heterologous enzyme molecules presented on its outer surface.

In some embodiments, according to any of the engineered OMVs described herein, the engineered OMV has a diameter of about 20 nm to about 500 nm, including, for example, about any of 30 nm, 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, 260 nm, 270 nm, 280 nm, 290 nm, 300 nm, 400 nm, and 500 nm, including any ranges between any of these values. For example, in some embodiments, the engineered OMV has a diameter of about 50 nm to about 300 nm, about 70 nm to about 200 nm, about 70 nm to about 130 nm, about 80 nm to about 120 nm, about 90 nm to about 110 nm, or about 100 nm.

In some embodiments, according to any of the engineered OMVs described herein, the engineered OMV is obtained from a photosynthetic microorganism. In some specific embodiments, the photosynthetic microorganism is a gram-negative bacterium. In yet other specific embodiments, the photosynthetic microorganism is a eukaryotic microalgae.

Thus, in some embodiments, provided herein is an engineered OMV according to any of the embodiments described here, wherein the engineered OMV is obtained from a gram-negative bacterium. In some embodiments, the gram-negative bacterium is a cyanobacterium. In some specific embodiments, the cyanobacterium is selected from Synechocystis sp., Synechococcus sp., Microcystis aeruginosa, Leptolyngbya boryana, Cyanobium gracile, Phormidium persicinum, and Gloeocapsa sp. In some embodiments, the cyanobacterium is selected from Synechococcus sp. PCC 7942, Synechococcus elongatus UTEX 2973, and Synechocystis sp. PCC 6803.

In some embodiments, according to any of the engineered OMVs described herein, the heterologous enzyme comprises a carbonic anhydrase (CA) polypeptide or functional fragment or derivative thereof. For example, in some embodiments, the heterologous enzyme comprises a polypeptide having a carbonic anhydrase activity. In some embodiments, the polypeptide having a carbonic anhydrase activity is derived from a thermostable carbonic anhydrase, such as a carbonic anhydrase from Thermosulfurimonas dismutans. In some embodiments, the polypeptide having a carbonic anhydrase activity is a functional fragment of a carbonic anhydrase from Thermosulfurimonas dismutans retaining at least some (e.g., at least about 50%, 60%, 70%, 80%, 90%, 91%, 92%, 93%, 94%, 95%, 96%, 97%, 98%, or 99%) of its carbonic anhydrase activity. In some embodiments, the polypeptide having a carbonic anhydrase activity is a functional derivative of a carbonic anhydrase from Thermosulfurimonas dismutans, such as a polypeptide having sequence similarity (e.g., at least about any of 70%, 80%, 90%, 91%, 92%, 93%, 94%, 95%, 96%, 97%, 98%, or 99% sequence identity) to a carbonic anhydrase from Thermosulfurimonas dismutans and retaining at least some (e.g., at least about 50%, 60%, 70%, 80%, 90%, 91%, 92%, 93%, 94%, 95%, 96%, 97%, 98%, or 99%) of its carbonic anhydrase activity. In some specific embodiments, the polypeptide having a carbonic anhydrase activity comprises the amino acid sequence of SEQ ID NO: 7, or a variant thereof having at least 90% sequence identity thereto.

In some embodiments, provided herein is a nanocatalyst composition comprising an engineered OMV according to any of the embodiments described herein. In some embodiments, the engineered OMV is produced by a method according to any of the embodiments described herein.

In some embodiments, according to any of the nanocatalyst compositions described herein, the nanocatalyst composition comprises the engineered OMV at a concentration greater than about 100 mg/L, including, for example, greater than about any of 200 mg/L, 300 mg/L, 400 mg/L, 500 mg/L, 600 mg/L, 700 mg/L, 800 mg/L, 900 mg/L, 1 g/L, 2 g/L, 3 g/L, 4 g/L, 5 g/L, 6 g/L, 7 g/L, 8 g/L, 9 g/L, 10 g/L, or greater.

In some embodiments, according to any of the nanocatalyst compositions described herein, the nanocatalyst composition comprises the engineered OMV in an amount greater than about 10 g, including, for example, greater than about any of 20 g, 30 g, 40 g, 50 g, 60 g, 70 g, 80 g, 90 g, 100 g, 200 g, 300 g, 400 g, 500 g, or greater.

In some embodiments, according to any of the nanocatalyst compositions described herein, the nanocatalyst composition is not a therapeutic composition. In some embodiments, according to any of the nanocatalyst compositions described herein, the nanocatalyst composition is not a composition of therapeutic quality. In some embodiments, according to any of the nanocatalyst compositions described herein, the nanocatalyst composition is a composition for industrial use.

In some embodiments, provided herein is a nanocatalyst product comprising a nanocatalyst composition according to any of the embodiments described herein. In some embodiments, the nanocatalyst product comprises the nanocatalyst composition in a volume greater than about 100 mL, including, for example, greater than about any of 200 mL, 300 mL, 400 mL, 500 mL, 600 mL, 700 mL, 800 mL, 900 mL, 1 L, 2 L, 3 L, 4 L, 5 L, or greater.

### C. Transmembrane Proteins

According to the present disclosure, one approach for presenting a heterologous enzyme on the outer surface of an OMV is to fuse the heterologous enzyme to a transmembrane protein to form a fusion protein in such a way that expression of the fusion protein in a host cell results in the production of OMVs with the fusion protein embedded in their membrane and oriented such that the heterologous enzyme is presented on the outer surface of the OMVs.

### D. Autotransporter Proteins (ATPs)

In some embodiments, the transmembrane domain is derived from an autotransporter protein (ATP). In some embodiments, the ATP is selected from antigen 43 (Ag43), hemoglobin-binding protease (Hbp), pertactin, extracellular serine protease plasmid-encoded (EspP), IgA1 protease, esterase autotransporter (EstA), adhesion and penetration protein (Hap), adhesin involved in diffuse adherence (AIDA-I), plasmid-encoded toxin (Pet), protease involved in intestinal colonization (Pic), temperature-sensitive hemagglutinin (Tsh), shigella extracellular protein (SepA), and vacuolating cytotoxin A (VacA).

In some embodiments, the transmembrane domain is derived from porins, ice-nucleation proteins (INP), or pilins.

### E. Outer Membrane Proteins (OMPs)

In some embodiments, the transmembrane domain is derived from an outer membrane protein (OMP). In some embodiments, the OMP is selected from Synechococcus outer membrane protein A (SomA), MipA, and ice nucleation proteins (INPs).

In some embodiments, the INP is a InaZ derived from Pseudomonas syringae. In some embodiments, the INP is derived from Erwinia herbicola or Xanthomonas campestris.

In some embodiments, the OMP is MipA.

In some embodiment, the OMP is a Ton-B-dependent transporter (TBDTs). In some embodiments, the TBDts are selected from ferric enterobactin transporter and vitamin B12 transporter.

### F. Carbonic Anhydrase

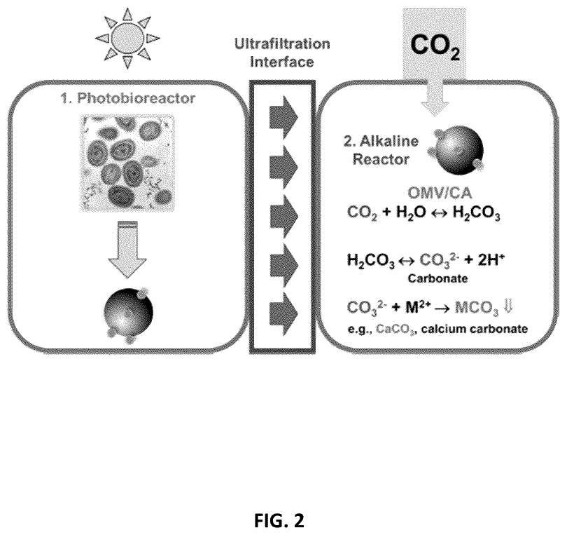

According to the present disclosure, the engineered OMVs may be employed to catalyze the conversion of CO₂ and H₂O to HCO₃⁻ and H⁺, a reaction catalyzed by the metalloenzyme carbonic anhydrase. A non-limiting example of this conversion is provided in FIG. 2.

In some embodiments, an engineered OMV according to any of the embodiments described herein comprises a fusion protein comprising a polypeptide have a carbonic anhydrase activity.

Carbonic anhydrases (CAs) are metalloenzymes that catalyze the conversion of CO₂ to bicarbonate ions and protons, which is used by cells to control pH and CO₂.

In some embodiments, the heterologous enzyme comprises a polypeptide having a carbonic anhydrase activity. In some embodiments, the polypeptide having a carbonic anhydrase activity is derived from a thermostable carbonic anhydrase, such as a carbonic anhydrase from Thermosulfurimonas dismutans. In some embodiments, the polypeptide having a carbonic anhydrase activity is a functional fragment of a carbonic anhydrase from Thermosulfurimonas dismutans retaining at least some (e.g., at least about 50%, 60%, 70%, 80%, 90%, 91%, 92%, 93%, 94%, 95%, 96%, 97%, 98%, or 99%) of its carbonic anhydrase activity. In some embodiments, the polypeptide having a carbonic anhydrase activity is a functional derivative of a carbonic anhydrase from Thermosulfurimonas dismutans, such as a polypeptide having sequence similarity (e.g., at least about any of 70%, 80%, 90%, 91%, 92%, 93%, 94%, 95%, 96%, 97%, 98%, or 99% sequence identity) to a carbonic anhydrase from Thermosulfurimonas dismutans and retaining at least some (e.g., at least about 50%, 60%, 70%, 80%, 90%, 91%, 92%, 93%, 94%, 95%, 96%, 97%, 98%, or 99%) of its carbonic anhydrase activity. In some specific embodiments, the polypeptide having a carbonic anhydrase activity comprises the amino acid sequence of SEQ ID NO: 7, or a variant thereof having at least 90% sequence identity thereto.

In some embodiments, a carbonic anhydrase (CA) can function at high temperatures (over 60° C.) from the bacterium Thermosulfurimonas dismutan.

In some embodiments, the carbonic anhydrase (CA) enzyme is selected from one or more of the following: aCA; (3CA; yCA; 6CA; (CA; r1CA; OCA; and LCA. In some embodiments, the CA enzyme is selected from one or more of: Gracilariopsis chorda CA (NBIV01000047.1); 004846, ATCA1_ARATH; F4IHR4, ATCA2_ARATH; Q9FYE3, ATCA3_ARATH; F4JI K2, ATCA4_ARATH; F4HUC4, ATCA5_ARATH; Q9SUB4, ATCA6_ARATH; Q8L817, ATCA7_ARATH; Q9FM99, ATCA8_ARATH; P27140, BCA1_ARATH; A8XKVO, BCA1_CAEBR; Q22460, BCA1_CAEEL; P42737, BCA2_ARATH; Q9ZUC2, BCA3_ARATH; Q94CE4, BCA4_ARATH; Q94CE3, BCA5_ARATH; Q9C6F5, BCA6_ARATH; 043570, CAH12_HUMAN; Q8CI85, CAH12_MOUSE; Q9MZ30, CAH12_RABIT; Q8NIQ1, CAH13_HUMAN; Q9D6N1, CAH13_MOUSE; Q9ULX7, CAH14_HUMAN; Q9WVT6, CAH14_MOUSE; Q99N23, CAH15_MOUSE; Q1LZA1, CAH1_BOVIN; P83299, CAH1_CHIHA; P20507, CAH1_CHLRE; P46512, CAH1_FLALI; Q7M316, CAH1_GORGO; P00917, CAH1_HORSE; P00915, CAH1_HUMAN; B3A0P2, CAH1_LOTGI; P00916, CAH1_MACMU; P35217, CAH1_MACNE; Q8HY33, CAH1_MONDO; P13634, CAH1_MOUSE; Q7M317, CAH1_PANTR; P07452, CAH1_RABIT; BOBNN3, CAH1_RAT; P48282, CAH1_SHEEP; P00921, CAH2_BOVIN; P07630, CAH2_CHICK; P24258, CAH2_CHLRE; P46513, CAH2_FLALI; P00918, CAH2_HUMAN; B3AOQ6, CAH2_LOTGI; P00920, CAH2_MOUSE; P00919, CAH2_RABIT; P27139, CAH2_RAT; P00922, CAH2_SHEEP; Q8UWA5, CAH2_TRIHK; Q3SZX4, CAH3_BOVIN; Q27504, CAH3 CAEEL; P07450, CAH3_HORSE; P07451, CAH3_HUMAN; P16015, CAH3_MOUSE; Q55154, CAH3_PIG; P14141, CAH3_RAT; Q95323, CAH4_BOVIN; P22748, CAH4_HUMAN; Q64444, CAH4_MOUSE; P48283, CAH4_RABIT; P48284, CAH4_RAT; P35218, CAHSA_HUMAN; P23589, CAHSA_MOUSE; P43165, CAHSA_RAT; Q9Y2DO, CAH5B_HUMAN; Q9QZAO, CAH5B_MOUSE; Q66HG6, CAH5B_RAT; Q10462, CAH5_CAEEL; P18915, CAH6_BOVIN; Q865CO3 CAH6_CANLF; P23280, CAH6_HUMAN; P18761, CAH6_MOUSE; P08060, CAH6_SHEEP; P43166, CAH7_HUMAN; Q9ERQ8, CAH7_MOUSE; Q16790, CAH9_HUMAN; Q8VHB5, CAH9_MOUSE; P40880, CAHC_HORVU; P17067, CAHC_PEA; P16016, CAHC_SPIOL; P27141, CAHC_TOBAC; P46510, CAHX_FLABI; P46511, CAHX_FLABR; P46281, CAHX_FLAPR; Q92051, CAHZ_DANRE; B8V7P3, CAH_ACRMI; P54212, CAH_DUNSA; 052535, CAH_KLEPN; P84537, CAH_LOBCS; Q57752, CAH_METJA; P40881, CAH_METTT; Q50940, CAH_NEIGO; P94170, CAH_NOSS1; Q6DAJ6, CAH_PECAS; 052538, CAH_PECCA; Q5AJ71, CAN_CANAL; P61517, CAN_ECOLI; Q5BCC5, CAN_EMENI; P45148, CAN_HAEIN; 094255, CAN_SCHPO; P61518, CAN_SHIFL; P53615, CAN_YEAST; Q8YYI3, CCMM_NOSS1; Q8DKB5, CCMM_THEVB; 085042, CSOCA_HALNC; Q31HD6, CSOCA_HYDCU; Q7TTT8, CSOCA_PARMW; Q7V6G1, CSOCA_PROMM; Q7V2C7, CSOCA_PROMP; POABFO, CYNT_EC057; POABE9, CYNT_ECOLI; Q9ZN54, CYNT_HELPJ; 024855, CYNT_HELPY; AOR566, CYNT_MYCS2; Q91262, CYNT_PSEAE; P83329, CYNT_STRTR; P27134, CYNT_SYNE7; Q54735, CYNT_SYNY3; Q75N34, DB3S_DIOPO; A7MAQ2, DIOA3_DIOJA; Q9NL38, MA66_PINMA; AOZSF6, MAC1_CRANI; AOZSF7, MAC2_CRANI; AOZSF2, MAF_PINFU; AOZSF3, MAM_PINMA; Q27908, MANA_PINFU; AOZSF4, MAP1_MIZYE; AOZSF5, MAP2_MIZYE; P64798, MTCA1_MYCBO; POWPJ6, MTCA1_MYCTO; P9WPJ7, MTCA1_MYCTU; P9WPJ8, MTCA2_MYCTO; POWPJ9, MTCA2_MYCTU; Q84UV8, NEC3_NICLS; 034872, YTIB_BACSU; NBIV01000047.1; and 006983, YVDA_BACSU. Non-limiting examples of calcium anhydrase enzymes can be found in Canadian Application Publication No.: CA3171797, which is hereby incorporated by reference in its entirety.

### G. Examples of Fusion Proteins

In some embodiments, provided herein are fusion proteins according to any of the embodiments described herein comprising a heterologous enzyme and a transmembrane protein useful for presentation of the heterologous enzyme on the outer surface of an OMV.

In some embodiments, the fusion protein comprises, from N-terminus to C-terminus, a polypeptide having carbonic anhydrase activity and a transmembrane protein derived from an autotransporter protein (ATP). In some embodiments, the polypeptide having carbonic anhydrase activity comprises the amino acid sequence of SEQ ID NO: 7, or a variant thereof having at least about 90%, 91%, 92%, 93%, 94%, 95%, 96%, 97%, 98%, or 99% sequence identity thereto. In some embodiments, the transmembrane protein comprises the amino acid sequence of SEQ ID NO: 8 or 9, or a variant thereof having at least about 90%, 91%, 92%, 93%, 94%, 95%, 96%, 97%, 98%, or 99% sequence identity thereto. In some embodiments, the transmembrane protein comprises the amino acid sequence of SEQ ID NO: 10, or a variant thereof having at least about 90%, 91%, 92%, 93%, 94%, 95%, 96%, 97%, 98%, or 99% sequence identity thereto. In some embodiments, the fusion protein further comprises a peptide linker connecting the polypeptide having carbonic anhydrase activity to the transmembrane protein. In some embodiments, the peptide linker comprises the amino acid sequence of SEQ ID NO: 17. In some embodiments, the fusion protein further comprises an N-terminal signal peptide capable of targeting the fusion protein to the inner membrane of a host cell. In some embodiments, the signal peptide comprises the amino acid sequence of any one of SEQ ID NOs: 13-15. In some embodiments, the signal peptide comprises the amino acid sequence of SEQ ID NO: 22.

Accordingly, in some embodiments, provided herein is a fusion protein comprising the amino acid sequence of SEQ ID NO: 1. In some embodiments, provided herein is a fusion protein comprising an amino acid sequence having at least about 70%, 75%, 80%, 85%, 90%, 95%, 96%, 97%, 98%, or 99% identity to SEQ ID NO: 1. In some embodiments, provided herein is a fusion protein comprising an amino acid having at least about 90%, 91%, 92%, 93%, 94%, 95%, 96%, 97%, 98%, or 99% identity to SEQ ID NO: 1.

In some other embodiments, provided herein is a fusion protein comprising the amino acid sequence of SEQ ID NO: 2. In some embodiments, provided herein is a fusion protein comprising an amino acid sequence having at least about 70%, 75%, 80%, 85%, 90%, 95%, 96%, 97%, 98%, or 99% identity to SEQ ID NO: 2. In some embodiments, provided herein is a fusion protein comprising an amino acid sequence having at least about 90%, 91%, 92%, 93%, 94%, 95%, 96%, 97%, 98%, or 99% identity to SEQ ID NO: 2.

In yet some other embodiments, provided herein is a fusion protein comprising the amino acid sequence of SEQ ID NO: 3. In some embodiments, provided herein is a fusion protein comprising an amino acid sequence at least about 70%, 75%, 80%, 85%, 90%, 95%, 96%, 97%, 98%, or 99% identity to SEQ ID NO: 3. In some embodiments, provided herein is a fusion protein comprising an amino acid sequence having at least about 90%, 91%, 92%, 93%, 94%, 95%, 96%, 97%, 98%, or 99% identity to SEQ ID NO: 3.

In some embodiments, the fusion protein comprises, from N-terminus to C-terminus, a transmembrane protein derived from an outer membrane protein (OMP) and a polypeptide having carbonic anhydrase activity. In some embodiments, the polypeptide having carbonic anhydrase activity comprises the amino acid sequence of SEQ ID NO: 7, or a variant thereof having at least about 90%, 91%, 92%, 93%, 94%, 95%, 96%, 97%, 98%, or 99% sequence identity thereto. In some embodiments, the transmembrane protein comprises the amino acid sequence of any one of SEQ ID NOs: 10-12, or a variant thereof having at least about 90%, 91%, 92%, 93%, 94%, 95%, 96%, 97%, 98%, or 99% sequence identity thereto. In some embodiments, the fusion protein further comprises a peptide linker connecting the polypeptide having carbonic anhydrase activity to the transmembrane protein. In some embodiments, the peptide linker comprises the amino acid sequence of SEQ ID NO: 18 or 19. In some embodiments, the fusion protein further comprises an N-terminal signal peptide capable of targeting the fusion protein to the inner membrane of a host cell. In some embodiments, the signal peptide comprises the amino acid sequence of SEQ ID NO: 16.

Accordingly, in some embodiments, provided herein is a fusion protein comprising the amino acid sequence of SEQ ID NO: 4. In some embodiments, provided herein is a fusion protein comprising an amino acid sequence having at least about 70%, 75%, 80%, 85%, 90%, 95%, 96%, 97%, 98%, or 99% identity to SEQ ID NO: 4. In some embodiments, provided herein is a fusion protein comprising an amino acid sequence having at least about 90%, 91%, 92%, 93%, 94%, 95%, 96%, 97%, 98%, or 99% identity to SEQ ID NO: 4.

In some other embodiments, provided herein is a fusion protein comprising the amino acid sequence of SEQ ID NO: 5. In some embodiments, provided herein is a fusion protein comprising an amino acid sequence having at least about 70%, 75%, 80%, 85%, 90%, 95%, 96%, 97%, 98%, or 99% identity to SEQ ID NO: 5. In some embodiments, provided herein is a fusion protein comprising an amino acid sequence having at least about 90%, 91%, 92%, 93%, 94%, 95%, 96%, 97%, 98%, or 99% identity to SEQ ID NO: 5.

In yet some other embodiments, provided herein is a fusion protein comprising the amino acid sequence of SEQ ID NO: 6. In some embodiments, provided herein is a fusion protein comprising an amino acid sequence having at least about 70%, 75%, 80%, 85%, 90%, 95%, 96%, 97%, 98%, or 99% identity to SEQ ID NO: 6. In some embodiments, provided herein is a fusion protein comprising an amino acid sequence having at least about 90%, 91%, 92%, 93%, 94%, 95%, 96%, 97%, 98%, or 99% identity to SEQ ID NO: 6.

In yet some other embodiments, provided herein is a fusion protein comprising the amino acid sequence of SEQ ID NO: 20. In some embodiments, provided herein is a fusion protein comprising an amino acid sequence having at least about 70%, 75%, 80%, 85%, 90%, 95%, 96%, 97%, 98%, or 99% identity to SEQ ID NO: 20. In some embodiments, provided herein is a fusion protein comprising an amino acid sequence having at least about 90%, 91%, 92%, 93%, 94%, 95%, 96%, 97%, 98%, or 99% identity to SEQ ID NO: 20.

In yet some other embodiments, provided herein is a fusion protein comprising the amino acid sequence of SEQ ID NO: 21. In some embodiments, provided herein is a fusion protein comprising an amino acid sequence having at least about 70%, 75%, 80%, 85%, 90%, 95%, 96%, 97%, 98%, or 99% identity to SEQ ID NO: 21. In some embodiments, provided herein is a fusion protein comprising an amino acid sequence having at least about 90%, 91%, 92%, 93%, 94%, 95%, 96%, 97%, 98%, or 99% identity to SEQ ID NO: 21.

In yet some other embodiments, provided herein is a nucleic acid encoding a fusion protein comprising the nucleotide sequence of SEQ ID NO: 24. In some embodiments, provided herein is a nucleic acid encoding a fusion protein comprising a nucleotide sequence having at least about 70%, 75%, 80%, 85%, 90%, 95%, 96%, 97%, 98%, or 99% identity to SEQ ID NO: 24. In some embodiments, provided herein is a nucleic acid encoding a fusion protein comprising a nucleotide sequence having at least about 90%, 91%, 92%, 93%, 94%, 95%, 96%, 97%, 98%, or 99% identity to SEQ ID NO: 21.

### H. Preparation of Engineered OMVs

Enzyme-decorated OMVs may be produced by culturing cells transformed or transfected with nucleic acid encoding the enzyme or a fusion protein comprising the enzyme. Polynucleotide sequences encoding polypeptide components of the engineered OMVs of the present disclosure can be obtained using standard recombinant techniques. In some embodiments, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells are transformed with the above-described expression vectors and cultured in conventional nutrient media modified as appropriate for inducing promoters, selecting transformants, or amplifying the genes encoding the desired sequences. OMVs produced by the host cells can be purified using standard purification methods as known in the art.

### I. Engineered Host Cells

In one aspect of the present disclosure, provided herein is an engineered host cell capable of producing an OMV comprising a heterologous enzyme presented on the outer surface of the OMV, wherein the engineered host cell is a photosynthetic microorganism comprising heterologous nucleic acid encoding a fusion protein comprising the heterologous enzyme operably linked to a transmembrane protein, and wherein the fusion protein is capable of being inserted into the outer membrane of the engineered host cell such that the heterologous enzyme is presented on the outer surface of the engineered host cell.

Thus, in some embodiments, provided herein is an engineered host cell capable of producing an OMV comprising a heterologous enzyme presented on the outer surface of the OMV, wherein the engineered host cell is a photosynthetic microorganism comprising heterologous nucleic acid encoding a fusion protein comprising the heterologous enzyme operably linked to a transmembrane protein, wherein the fusion protein is capable of being inserted into the outer membrane of the engineered host cell such that the heterologous enzyme is presented on the outer surface of the engineered host cell, and wherein the transmembrane protein is derived from an ATP. In some embodiments, the ATP is selected from antigen 43 (Ag43), hemoglobin-binding protease (Hbp), pertactin, extracellular serine protease plasmid-encoded (EspP), IgA1 protease, esterase autotransporter (EstA), adhesion and penetration protein (Hap), adhesin involved in diffuse adherence (AIDA-I), plasmid-encoded toxin (Pet), protease involved in intestinal colonization (Pic), temperature-sensitive hemagglutinin (Tsh), shigella extracellular protein (SepA), and vacuolating cytotoxin A (VacA). In some specific embodiments, the ATP is Ag43. In some such embodiments, the transmembrane protein comprises the amino acid sequence of SEQ ID NO: 8, or a variant thereof having at least about 90%, 91%, 92%, 93%, 94%, 95%, 96%, 97%, 98%, or 99% sequence identity thereto. In yet other specific embodiments, the ATP is Hbp. In some such embodiments, the transmembrane protein comprises the amino acid sequence of SEQ ID NO: 9, or a variant thereof having at least about 90%, 91%, 92%, 93%, 94%, 95%, 96%, 97%, 98%, or 99% sequence identity thereto.

In some other embodiments, provided herein is an engineered host cell capable of producing an OMV comprising a heterologous enzyme presented on the outer surface of the OMV, wherein the engineered host cell is a photosynthetic microorganism comprising heterologous nucleic acid encoding a fusion protein comprising the heterologous enzyme operably linked to a transmembrane protein, wherein the fusion protein is capable of being inserted into the outer membrane of the engineered host cell such that the heterologous enzyme is presented on the outer surface of the engineered host cell, and wherein the transmembrane protein is derived from an OMP selected from Synechococcus outer membrane protein A (SomA), MipA, and ice nucleation proteins (INPs). In some specific embodiments, the OMP is SomA. In some such embodiments, the transmembrane protein comprises the amino acid sequence of SEQ ID NO: 10, or a variant thereof having at least about 90%, 91%, 92%, 93%, 94%, 95%, 96%, 97%, 98%, or 99% sequence identity thereto. In yet other specific embodiments, the OMP is MipA. In some such embodiments, the transmembrane protein comprises the amino acid sequence of SEQ ID NO: 11, or a variant thereof having at least about 90%, 91%, 92%, 93%, 94%, 95%, 96%, 97%, 98%, or 99% sequence identity thereto. In yet other specific embodiments, the OMP is an INP. In some such embodiments, the INP is from Pseudomonas syringae. In some embodiments, the transmembrane protein comprises the amino acid sequence of SEQ ID NO: 12, or a variant thereof having at least about 90%, 91%, 92%, 93%, 94%, 95%, 96%, 97%, 98%, or 99% sequence identity thereto.

In some embodiments, according to any of the engineered host cells described herein, the fusion protein further comprises a linker connecting the heterologous enzyme to the transmembrane protein. In some embodiments, the linker is a peptide linker comprising the amino acid sequence of any one of SEQ ID NOs: 17-19 and 25.

In some embodiments, according to any of the engineered host cells described herein, the fusion protein further comprises a signal peptide capable of targeting the fusion protein to the inner membrane of the engineered host cell. In some embodiments, the signal peptide comprises the amino acid sequence of any one of SEQ ID NOs: 13-16.

In some embodiments, according to any of the engineered host cells described herein, the engineered host cell is a gram-negative bacterium. In some embodiments, the gram-negative bacterium is a cyanobacterium. In some specific embodiments, the cyanobacterium is selected from Synechocystis sp., Synechococcus sp., Microcystis aeruginosa, Leptolyngbya boryana, Cyanobium gracile, Phormidium persicinum, and Gloeocapsa sp. In some embodiments, the cyanobacterium is selected from Synechococcus sp. PCC 7942, Synechococcus elongatus UTEX 2973, and Synechocystis sp. PCC 6803.

In some embodiments, according to any of the engineered host cells described herein, the engineered host cell is a eukaryotic microalgae. In some embodiments, the microalgae is selected from one or more of: Dunaliella: Tetraselmis: Chlorella; and Chlamydomonas. The microalgae may be selected from one or more of: Chlorella vulgaris: Thalassiosira pseudonana: Isochrysis sp.; Nannochloropsis sp.; Tetraselmis sp.; Chaetoceros muelleri; Thalassiosira weissflogii; Pavlova sp.; Karenia brevis; Skeletonema sp.; Chaetoceros grad is; Chaetoceros calcitrans; Haematococcus pluvialis; Spirulina sp.; Chlamydomonas sp.; Chlorella sp.; Euhalothece sp. (alkalihalophilic cyanobacterium).

In some embodiments, according to any of the engineered host cells described herein, the heterologous enzyme comprises a carbonic anhydrase (CA) polypeptide or functional fragment or derivative thereof. For example, in some embodiments, the heterologous enzyme comprises a polypeptide having a carbonic anhydrase activity. In some embodiments, the polypeptide having a carbonic anhydrase activity is derived from a thermostable carbonic anhydrase, such as a carbonic anhydrase from Thermosulfurimonas dismutans. In some embodiments, the polypeptide having a carbonic anhydrase activity is a functional fragment of a carbonic anhydrase from Thermosulfurimonas dismutans retaining at least some (e.g., at least about 50%, 60%, 70%, 80%, 90%, 91%, 92%, 93%, 94%, 95%, 96%, 97%, 98%, or 99%) of its carbonic anhydrase activity. In some embodiments, the polypeptide having a carbonic anhydrase activity is a functional derivative of a carbonic anhydrase from Thermosulfurimonas dismutans, such as a polypeptide having sequence similarity (e.g., at least about any of 70%, 80%, 90%, 91%, 92%, 93%, 94%, 95%, 96%, 97%, 98%, or 99% sequence identity) to a carbonic anhydrase from Thermosulfurimonas dismutans and retaining at least some (e.g., at least about 50%, 60%, 70%, 80%, 90%, 91%, 92%, 93%, 94%, 95%, 96%, 97%, 98%, or 99%) of its carbonic anhydrase activity. In some specific embodiments, the polypeptide having a carbonic anhydrase activity comprises the amino acid sequence of SEQ ID NO: 7, or a variant thereof having at least 90% sequence identity thereto.

In some embodiments, according to any of the engineered host cells described herein, the heterologous nucleic acid is present extrachromosomally. In some embodiments, the heterologous nucleic acid is a plasmid.

In some embodiments, according to any of the engineered host cells described herein, the heterologous nucleic acid is integrated into the chromosome of the engineered host cell. In some embodiments, the heterologous nucleic acid is integrated into the chromosome by site-specific targeting. In some embodiments, the heterologous nucleic acid is integrated into the chromosome by site-specific targeting using a CRISPR/Cas9 system.

In some embodiments, according to any of the engineered host cells described herein, the heterologous nucleic acid is operably linked to an inducible promoter.

Accordingly, in some embodiments, provided herein is a method of producing an engineered OMV comprising (a) culturing an engineered host cell according to any one of the embodiments described herein under conditions suitable for expression of the fusion protein; and (b) obtaining an OMV produced by the engineered host cell, thereby producing the engineered OMV. In some embodiments, he engineered host cell is cultured in the presence of CO2 and light. In some embodiments, the method further comprises filtering the engineered OMV. In some embodiments, the filtering is performed by ultrafiltration. In some embodiments, the filtering is performed by a membrane bioreactor. In some embodiments, the method further comprises concentrating the engineered OMV.

### J. Manufacturing Systems

In one aspect of the present disclosure, provided herein is a system capable of producing an engineered OMV according to any of the embodiments described herein. In some embodiments, the system is capable of producing a composition comprising a plurality of the engineered OMVs. In some embodiments, the composition is suitable for use in an industrial application. For example, in some embodiments, the composition (a) comprises the engineered OMV at a concentration greater than about 100 mg/L (including, for example, greater than about any of 200 mg/L, 300 mg/L, 400 mg/L, 500 mg/L, 600 mg/L, 700 mg/L. 800 mg/L. 900 mg/L, 1 g/L, 2 g/L, 3 g/L, 4 g/L, 5 g/L, 6 g/L, 7 g/L, 8 g/L, 9 g/L, 10 g/L, or greater): (b) comprises the engineered OMV in an amount greater than about 10 g (including, for example, greater than about any of 20 g. 30 g. 40 g. 50 g, 60 g. 70 g, 80 g, 90 g, 100 g. 200 g. 300 g. 400 g. 500 g, or greater); and/or (c) has a volume greater than about 100 mL (including, for example, greater than about any of 200 mL, 300 mL, 400 mL, 500 mL, 600 mL, 700 mL, 800 mL, 900 mL, 1 L, 2 L, 3 L, 4 L, 5 L, or greater).

Thus, in some embodiments, provided herein is a system capable of producing a composition comprising an engineered OMV according to any of the embodiments described herein. In some embodiments, the system comprises a first reactor (e.g., a photobioreactor) for the production of the engineered OMV. In some embodiments, the first reactor is a photobioreactor configured to receive light and photosynthetic microorganisms (e.g., cyanobacteria or eukaryotic microalgae) capable of producing the engineered OMV, wherein culturing the photosynthetic microorganisms under suitable conditions (e.g., appropriate light exposure, culture medium, and incubation conditions, such as temperature and agitation) allows for the production of the engineered OMV in the photobioreactor. In some embodiments, the photobioreactor is configured to receive ambient air/CO2. In some embodiments, the system further comprises a receptacle configured to receive engineered OMVs produced in the photobioreactor. In some embodiments, the receptacle is connected to the photobioreactor by way of a filtration interface (e.g., an ultrafiltration interface), wherein engineered OMVs produced in the photobioreactor are capable of passing through the filtration interface into the receptacle. In some embodiments, photosynthetic microorganisms in the photobioreactor are incapable or substantially incapable of passing through the filtration interface into the receptacle. In some embodiments, the filtration interface comprises one or more filtration membranes (e.g., ultrafiltration membranes). In some embodiments, the filtration interface comprises one or more (including, for example, 2, 3, 4, 5, 6, 7, 8, 9, 10, or more) ultrafiltration membranes.

In some embodiments, provided herein is a system for the production of an engineered OMV according to any of the embodiments described herein, the system comprising: (a) a first reactor configured to receive a microorganism capable of producing the engineered OMV; (b) a receptacle configured to receive the engineered OMV produced in the first reactor; and (c) a filtration interface connecting the first reactor to the receptable, wherein the engineered OMV produced in the first reactor is capable of passing through the filtration interface into the receptacle. In some embodiments, the first reactor is a photobioreactor configured to receive light, and the microorganism is a photosynthetic microorganism. In some embodiments, the photosynthetic microorganism is a cyanobacterium or a eukaryotic microalga. In some embodiments, the photosynthetic microorganism is an engineered host cell according to any of the embodiments described herein. In some embodiments, the receptable is a second reactor. In some embodiments, the second reactor is in conditions suitable for carrying out the conversion of CO₂ and H₂O to HCO₃⁻ and H⁺.

In some embodiments, according to any of the systems for the production of an engineered OMV described herein, the first reactor is configured to receive ambient air/CO2.

In some embodiments, according to any of the systems for the production of an engineered OMV described herein, the microorganism in the first reactor is incapable or substantially incapable of passing through the filtration interface into the receptacle.

In some embodiments, according to any of the systems for the production of an engineered OMV described herein, the filtration interface comprises one or more filtration membranes. In some embodiments, the filtration interface comprises at least 2, 3, 4, or 5 filtration membranes. In some embodiments, the one or more filtration membranes are one or more ultrafiltration membranes. In some embodiments, the filtration interface comprises at least 2, 3, 4, or 5 ultrafiltration membranes.

In some embodiments, according to any of the systems for the production of an engineered OMV described herein, the receptacle is configured to receive the engineered OMV: (a) at a concentration greater than about 100 mg/L (including, for example, greater than about any of 200 mg/L, 300 mg/L, 400 mg/L, 500 mg/L, 600 mg/L, 700 mg/L, 800 mg/L, 900 mg/L, 1 g/L, 2 g/L, 3 g/L, 4 g/L, 5 g/L, 6 g/L, 7 g/L, 8 g/L, 9 g/L, 10 g/L, or greater): (b) in an amount greater than about 10 g (including, for example, greater than about any of 20 g, 30 g, 40 g, 50 g, 60 g, 70 g, 80 g, 90 g, 100 g, 200 g, 300 g, 400 g, 500 g, or greater); and/or (c) in a volume greater than about 100 mL (including, for example, greater than about any of 200 mL, 300 mL, 400 mL, 500 mL, 600 mL, 700 mL, 800 mL, 900 mL, 1 L, 2 L, 3 L, 4 L, 5 L, or greater).

### K. Methods of Using the Engineered OMVs

As would be appreciated from the present disclosure, the engineered OMVs according to the present disclosure can be used for a variety of purposes.

Accordingly, in one aspect, provided herein is a method of reducing CO₂ comprising interacting an engineered OMV according to any of the embodiments described herein employing a fusion protein comprising a polypeptide having a carbonic anhydrase activity with CO₂ under a condition suitable for the polypeptide having a carbonic anhydrase activity to convert CO₂ to a catalysis product. In some embodiments, the catalysis product is a metal carbonate. In some embodiments, the engineered OMV is interacted with CO₂ in the presence of H₂O and a metal ion under alkaline conditions suitable for (i) the conversion of CO₂ to CO₃²⁻ by the polypeptide having a carbonic anhydrase activity, and (ii) association of CO₃²⁻ with the metal ion to form the metal carbonate. In some embodiments, the metal ion is a divalent metal cation. In some embodiments, the metal ion is Ca²⁺, Mg²⁺, Fe²⁺, Zn²⁺, Cu²⁺, or Mn²⁺. Accordingly, in some embodiments, the catalysis product is CaCO₃, MgCO₃, FeCO₃, ZnCO₃, CuCO₃, or MnCO₃. In some embodiments, the metal ion is a monovalent metal cation. In some embodiments, the metal ion is Li⁺. Accordingly, in some embodiments, the catalysis product is Li₂CO₃.

In some embodiments, the engineered OMV catalyzes the reversable reaction CO₂+H₂O═H₂CO₃ (carbonic acid: subsequently forming HCO₃ [bicarbonate]+H⁺). In some embodiment, the reaction is performed under mildly alkaline condition (e.g., pH 10) and in the presence of a suitable salt (e.g., CaCl₂)). In some embodiments, the bicarbonate precipitates spontaneously as an insoluble carbonate (e.g., calcium carbonate, CaCO₃). In some embodiments, the engineered OMV is used to capture dissolved CO₂ as stable carbonate solids. The process can be used for carbon capture, storage, and utilization. (FIG. 2).

In some embodiments, according to any of the methods of reducing CO2 described herein, the engineered OMV is interacted with CO2 in air, water, or waste.

In some embodiments, according to any of the methods of reducing CO2 described herein, the method further comprises producing the engineered OMV according to any of the method described herein. In some embodiments, producing the engineered OMV is carried out in a first reactor and producing the catalysis product is carried out in a second reactor. In some embodiments, the method further comprises isolating engineered OMVs produced in the first reactor and introducing the isolated engineered OMVs into the second reactor. In some embodiments, isolating the engineered OMVs is carried out by filtration.

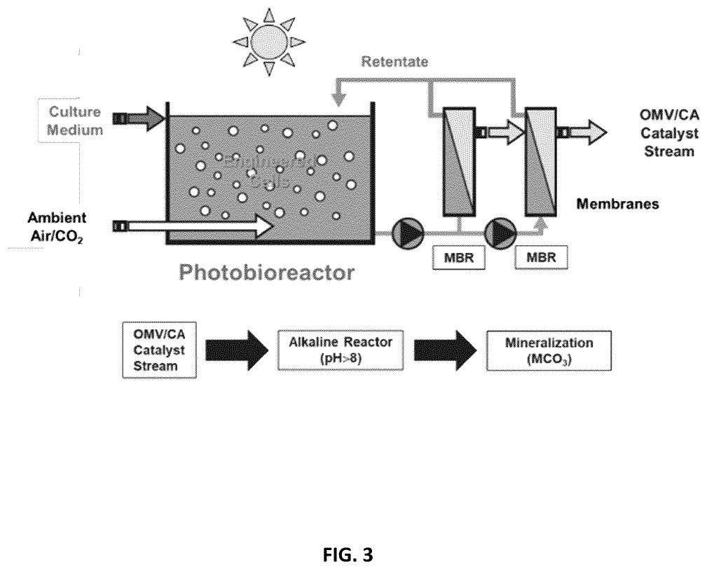

In some embodiments, the method of separating the engineered OMVs are scaled up for industrial application. In some embodiments, the system illustrated in FIG. 3 is used.

### L. Reactor Systems

In one aspect of the present disclosure, provided herein is a system capable of producing an engineered OMV according to any of the embodiments described herein and carrying out a reaction using the engineered OMV. In some embodiments, the engineered OMV presents on its surface a polypeptide having a carbonic anhydrase activity, and the engineered OMV is used to catalyze the conversion of CO₂ and H₂O to HCO₃⁻ and H⁺. In some embodiments, the system is capable of producing a composition comprising a plurality of the engineered OMVs. In some embodiments, the composition (a) comprises the engineered OMV at a concentration greater than about 100 mg/L (including, for example, greater than about any of 200 mg/L, 300 mg/L, 400 mg/L, 500 mg/L, 600 mg/L, 700 mg/L, 800 mg/L, 900 mg/L, 1 g/L, 2 g/L, 3 g/L, 4 g/L, 5 g/L, 6 g/L, 7 g/L, 8 g/L, 9 g/L, 10 g/L, or greater); (b) comprises the engineered OMV in an amount greater than about 10 g (including, for example, greater than about any of 20 g, 30 g, 40 g, 50 g, 60 g, 70 g, 80 g, 90 g, 100 g, 200 g, 300 g, 400 g, 500 g, or greater); and/or (c) has a volume greater than about 100 mL (including, for example, greater than about any of 200 mL, 300 mL, 400 mL, 500 mL, 600 mL, 700 mL, 800 mL, 900 mL, 1 L, 2 L, 3 L, 4 L, 5 L, or greater).

Thus, in some embodiments, provided herein is a system capable of catalyzing the conversion of CO₂ and H₂O to HCO₃⁻ and H⁺ using an engineered OMV according to any of the embodiments described herein presenting on its surface a polypeptide having a carbonic anhydrase activity. In some embodiments, the system comprises a first reactor (e.g., a photobioreactor) for the production of the engineered OMV. In some embodiments, the first reactor is a photobioreactor configured to receive light and photosynthetic microorganisms (e.g., cyanobacteria or eukaryotic microalgae) capable of producing the engineered OMV, wherein culturing the photosynthetic microorganisms under suitable conditions (e.g., appropriate light exposure, culture medium, and incubation conditions, such as temperature and agitation) allows for the production of the engineered OMV in the photobioreactor. In some embodiments, the photobioreactor is configured to receive ambient air/CO₂. In some embodiments, the system further comprises a second reactor configured to receive engineered OMVs produced in the photobioreactor into an aqueous composition and carry out the conversion of CO₂ and H₂O to HCO₃⁻ and H⁺. In some embodiments, the second reactor is an alkaline reactor, e.g., a reactor comprising an aqueous composition having a pH greater than about 8.0 (including, for example, greater than about any of 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or greater). In some embodiments, the second reactor is configured to receive CO₂. In some embodiments, the aqueous composition in the second reactor further comprises a metal ion. In some embodiments, the metal ion is a divalent metal cation. In some embodiments, the metal ion is Ca²⁺, Mg²⁺, Fe²⁺, Zn²⁺, Cu²⁺, or Mn²⁺. In some embodiments, the metal ion is a monovalent metal cation. In some embodiments, the metal ion is Li⁺. Accordingly, in some embodiments, a metal carbonate (e.g., CaCO₃, MgCO₃, FeCO₃, ZnCO₃, CuCO₃, MnCO₃, or Li₂CO₃) is formed in the second reactor. In some embodiments, the second reactor is connected to the photobioreactor by way of a filtration interface (e.g., an ultrafiltration interface), wherein engineered OMVs produced in the photobioreactor are capable of passing through the filtration interface into the second reactor. In some embodiments, photosynthetic microorganisms in the photobioreactor are incapable or substantially incapable of passing through the filtration interface into the second reactor. In some embodiments, the filtration interface comprises one or more filtration membranes (e.g., ultrafiltration membranes). In some embodiments, the filtration interface comprises one or more (including, for example, 2, 3, 4, 5, 6, 7, 8, 9, 10, or more) ultrafiltration membranes.

In some embodiments, provided herein is a system for catalyzing the conversion of CO₂ and H₂O to HCO₃⁻ and H⁺, the system comprising: (a) a first reactor configured to receive a microorganism capable of producing an engineered OMV according to any of the embodiments described herein presenting a heterologous enzyme comprising a carbonic anhydrase (CA) polypeptide or functional fragment thereof: (b) a second reactor configured to receive the engineered OMV produced in the first reactor in conditions suitable for carrying out the conversion of CO₂ and H₂O to HCO₃⁻ and H⁺; and (c) a filtration interface connecting the first reactor to the second reactor, wherein the engineered OMV produced in the first reactor is capable of passing through the filtration interface into the second reactor. In some embodiments, the first reactor is a photobioreactor configured to receive light, and the microorganism is a photosynthetic microorganism. In some embodiments, the photosynthetic microorganism is a cyanobacterium or a eukaryotic microalga. In some embodiments, the photosynthetic microorganism is an engineered host cell according to any of the embodiments described herein.

In some embodiments, according to any of the systems for catalyzing the conversion of CO₂ and H₂O to HCO₃⁻ and H⁺ described herein, the first reactor is configured to receive ambient air/CO₂.

In some embodiments, according to any of the systems for catalyzing the conversion of CO₂ and H₂O to HCO₃⁻ and H⁺ described herein, the second reactor is an alkaline reactor comprising an aqueous composition having a pH greater than about 8.0 (including, for example, greater than about any of 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, or greater).

In some embodiments, according to any of the systems for catalyzing the conversion of CO₂ and H₂O to HCO₃⁻ and H⁺ described herein, the second reactor is configured to receive CO₂.

In some embodiments, according to any of the systems for catalyzing the conversion of CO₂ and H₂O to HCO₃⁻ and H⁺ described herein, the second reactor comprises an aqueous composition comprising a metal ion under conditions suitable for the formation of a metal carbonate comprising the metal ion. In some embodiments, the metal ion is a divalent metal cation. In some embodiments, the metal ion is a monovalent metal cation. In some embodiments, the metal ion is Ca²⁺, Mg²⁺, Fe²⁺, Zn²⁺, Cu²⁺, Mn²⁺, or Li⁺. Accordingly, in some embodiments, the metal carbonate is CaCO₃, MgCO₃, FeCO₃, ZnCO₃, CuCO₃, MnCO₃, or Li₂CO₃.

In some embodiments, according to any of the systems for catalyzing the conversion of CO₂ and H₂O to HCO₃⁻ and H⁺ described herein, the microorganism in the first reactor is incapable or substantially incapable of passing through the filtration interface into the second reactor.

In some embodiments, according to any of the systems for catalyzing the conversion of CO₂ and H₂O to HCO₃⁻ and H⁺ described herein, the filtration interface comprises one or more filtration membranes. In some embodiments, the filtration interface comprises at least 2, 3, 4, or 5 filtration membranes. In some embodiments, the one or more filtration membranes are one or more ultrafiltration membranes. In some embodiments, the filtration interface comprises at least 2, 3, 4, or 5 ultrafiltration membranes.

In some embodiments, according to any of the systems for catalyzing the conversion of CO₂ and H₂O to HCO₃⁻ and H⁺ described herein, the second reactor is configured to receive the engineered OMV: (a) at a concentration greater than about 100 mg/L (including, for example, greater than about any of 200 mg/L, 300 mg/L, 400 mg/L, 500 mg/L, 600 mg/L, 700 mg/L, 800 mg/L, 900 mg/L, 1 g/L, 2 g/L, 3 g/L, 4 g/L, 5 g/L, 6 g/L, 7 g/L, 8 g/L, 9 g/L, 10 g/L, or greater); (b) in an amount greater than about 10 g (including, for example, greater than about any of 20 g, 30 g, 40 g, 50 g, 60 g, 70 g, 80 g, 90 g, 100 g, 200 g, 300 g, 400 g, 500 g, or greater); and/or (c) in a volume greater than about 100 mL (including, for example, greater than about any of 200 mL, 300 mL, 400 mL, 500 mL, 600 mL, 700 mL, 800 mL, 900 mL, 1 L, 2 L, 3 L, 4 L, 5 L, or greater).

### M. Kits

Also provided herein are kits comprising a nucleic acid encoding a fusion protein as provided herein, or a composition thereof, packaged into suitable packaging material. A kit optionally includes a label or packaging insert including a description of the components or instructions for use of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD-or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain nucleic acids provided herein, or cells that contain the nucleic acids. The cells in the kit can be maintained under appropriate storage conditions until ready to use.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

All applications, publications, patents and other references, GenBank citations and ATCC citations cited herein are incorporated by reference in their entirety. In case of conflict, the specification, including definitions, will control.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges includes combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys(C) | |
| glutamic acidGlu | (E) | |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

### 7 Non-Limiting Aspects and Embodiments of the Disclosure

Aspects, including embodiments, of the present subject matter described above may be beneficial alone or in combination, with one or more other aspects or embodiments. Without limiting the foregoing description, certain non-limiting aspects of the disclosure numbered 1-110 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. An engineered outer membrane vesicle (OMV) comprising a heterologous enzyme presented on the outer surface of the engineered OMV.

Aspect 2. The engineered OMV of aspect 1, wherein the engineered OMV is obtained from a photosynthetic microorganism.

Aspect 3. The engineered OMV of aspect 2, wherein the photosynthetic microorganism is an engineered photosynthetic microorganism.

Aspect 4. The engineered OMV of any one of aspects 2-3, wherein the photosynthetic microorganism is a gram-negative bacterium.

Aspect 5. The engineered OMV of aspect 4, wherein the gram-negative bacterium is a cyanobacterium.

Aspect 6. The engineered OMV of aspect 5, wherein the cyanobacterium is selected from Synechocystis sp., Synechococcus sp., Microcystis aeruginosa, Leptolyngbya boryana, Cyanobium gracile, Phormidium persicinum, and Gloeocapsa sp.

Aspect 7. The engineered OMV of aspect 5, wherein the cyanobacterium is selected from Synechococcus sp. PCC 7942, Synechococcus elongatus UTEX 2973, and Synechocystis sp. PCC 6803.

Aspect 8. The engineered OMV of any one of aspects 2-3, wherein the photosynthetic microorganism is a eukaryotic microalgae.

Aspect 9. The engineered OMV of any one of aspects 1-8, wherein the heterologous enzyme is operably linked to a transmembrane protein embedded in the membrane of the engineered OMV to form a fusion protein.

Aspect 10. The engineered OMV of aspect 9, wherein the transmembrane protein is derived from an autotransporter protein (ATP) or an outer membrane protein (OMP).

Aspect 11. The engineered OMV of aspect 10, wherein the transmembrane protein is derived from an ATP selected from antigen 43 (Ag43), hemoglobin-binding protease (Hbp), pertactin, extracellular serine protease plasmid-encoded (EspP), IgA1 protease, esterase autotransporter (EstA), adhesion and penetration protein (Hap), adhesin involved in diffuse adherence (AIDA-I), plasmid-encoded toxin (Pet), protease involved in intestinal colonization (Pic), temperature-sensitive hemagglutinin (Tsh), shigella extracellular protein (SepA), and vacuolating cytotoxin A (VacA).

Aspect 12. The engineered OMV of aspect 11, wherein the ATP is Ag43.

Aspect 13. The engineered OMV of aspect 12, wherein the transmembrane protein comprises the amino acid sequence of SEQ ID NO: 8.

Aspect 14. The engineered OMV of aspect 11, wherein the ATP is Hbp.

Aspect 15. The engineered OMV of aspect 14, wherein the transmembrane protein comprises the amino acid sequence of SEQ ID NO: 9.

Aspect 16. The engineered OMV of aspect 10, wherein the transmembrane protein is derived from an OMP selected from Synechococcus outer membrane protein A (SomA), MipA, and ice nucleation proteins (INPs).

Aspect 17. The engineered OMV of aspect 16, wherein the OMP is SomA.

Aspect 18. The engineered OMV of aspect 17, wherein the transmembrane protein comprises the amino acid sequence of SEQ ID NO: 10.

Aspect 19. The engineered OMV of aspect 16, wherein the OMP is MipA.

Aspect 20. The engineered OMV of aspect 19, wherein the transmembrane protein comprises the amino acid sequence of SEQ ID NO: 11.

Aspect 21. The engineered OMV of aspect 16, wherein the OMP is an INP.

Aspect 22. The engineered OMV of aspect 21, wherein the INP is from Pseudomonas syringae.

Aspect 23. The engineered OMV of aspect 22, wherein the transmembrane protein comprises the amino acid sequence of SEQ ID NO: 12.

Aspect 24. The engineered OMV of any one of aspects 9-23, wherein the fusion protein further comprises a linker connecting the heterologous enzyme to the transmembrane protein.

Aspect 25. The engineered OMV of aspect 17, wherein the linker is a peptide linker comprising the amino acid sequence of any one of SEQ ID NOs: 17-19 and 25.

Aspect 26. The engineered OMV of any one of aspects 1-25, wherein the engineered OMV comprises at least about 10 heterologous enzyme molecules presented on its outer surface.

Aspect 27. The engineered OMV of any one of aspects 1-26, wherein the engineered OMV has a diameter of about 50 nm to about 250 nm.

Aspect 28. The engineered OMV of aspect 27, wherein the engineered OMV has a diameter of about 70 nm to about 130 nm.

Aspect 29. The engineered OMV of any one of aspects 1-28, wherein the heterologous enzyme comprises a carbonic anhydrase (CA) polypeptide or functional fragment thereof.

Aspect 30. The engineered OMV of aspect 29, wherein the CA polypeptide is derived from a thermostable CA.

Aspect 31. The engineered OMV of aspect 30, wherein the thermostable CA is from Thermosulfurimonas dismutans.

Aspect 32. The engineered OMV of aspect 31, wherein the CA polypeptide comprises the amino acid sequence of SEQ ID NO: 7.

Aspect 33. An engineered host cell capable of producing an OMV comprising a heterologous enzyme presented on the outer surface of the OMV, wherein the engineered host cell is a photosynthetic microorganism comprising heterologous nucleic acid encoding a fusion protein comprising the heterologous enzyme operably linked to a transmembrane protein, and wherein the fusion protein is capable of being inserted into the outer membrane of the engineered host cell such that the heterologous enzyme is presented on the outer surface of the engineered host cell.

Aspect 34. The engineered host cell of aspect 33, wherein the transmembrane protein is derived from an autotransporter protein (ATP) or an outer membrane protein (OMP).

Aspect 35. The engineered host cell of aspect 34, wherein the transmembrane protein is derived from an ATP selected from antigen 43 (Ag43), hemoglobin-binding protease (Hbp), pertactin, extracellular serine protease plasmid-encoded (EspP), IgA1 protease, esterase autotransporter (EstA), adhesion and penetration protein (Hap), adhesin involved in diffuse adherence (AIDA-I), plasmid-encoded toxin (Pet), protease involved in intestinal colonization (Pic), temperature-sensitive hemagglutinin (Tsh), shigella extracellular protein (SepA), and vacuolating cytotoxin A (VacA).

Aspect 36. The engineered host cell of aspect 35, wherein the ATP is Ag43.

Aspect 37. The engineered host cell of aspect 36, wherein the transmembrane protein comprises the amino acid sequence of SEQ ID NO: 8.

Aspect 38. The engineered host cell of aspect 35, wherein the ATP is Hbp.

Aspect 39. The engineered host cell of aspect 38, wherein the transmembrane protein comprises the amino acid sequence of SEQ ID NO: 9.

Aspect 40. The engineered host cell of aspect 34, wherein the transmembrane protein is derived from an OMP selected from Synechococcus outer membrane protein A (SomA), MipA, and ice nucleation proteins (INPs).

Aspect 41. The engineered host cell of aspect 40, wherein the OMP is SomA.

Aspect 42. The engineered host cell of aspect 41, wherein the transmembrane protein comprises the amino acid sequence of SEQ ID NO: 10.

Aspect 43. The engineered host cell of aspect 40, wherein the OMP is MipA.

Aspect 44. The engineered host cell of aspect 43, wherein the transmembrane protein comprises the amino acid sequence of SEQ ID NO: 11.

Aspect 45. The engineered host cell of aspect 40, wherein the OMP is an INP.

Aspect 46. The engineered host cell of aspect 45, wherein the INP is from Pseudomonas syringae.

Aspect 47. The engineered host cell of aspect 46, wherein the transmembrane protein comprises the amino acid sequence of SEQ ID NO: 12.

Aspect 48. The engineered host cell of any one of aspects 33-47, wherein the fusion protein further comprises a linker connecting the heterologous enzyme to the transmembrane protein.

Aspect 49. The engineered host cell of aspect 48, wherein the linker is a peptide linker comprising the amino acid sequence of any one of SEQ ID NOs: 17-19 and 25.

Aspect 50. The engineered host cell of any one of aspects 33-49, wherein the fusion protein further comprises a signal peptide capable of targeting the fusion protein to the inner membrane of the engineered host cell.

Aspect 51. The engineered host cell of aspect 50, wherein the signal peptide comprises the amino acid sequence of any one of SEQ ID NOs: 13-16.

Aspect 52. The engineered host cell of any one of aspects 33-51, wherein the engineered host cell is a cyanobacterium.

Aspect 53. The engineered host cell of aspect 52, wherein the cyanobacterium is selected from Synechocystis sp., Synechococcus sp., Microcystis aeruginosa, Leptolyngbya boryana, Cyanobium gracile, Phormidium persicinum, and Gloeocapsa sp.

Aspect 54. The engineered host cell of aspect 53, wherein the cyanobacterium is selected from Synechococcus sp. PCC 7942, Synechococcus elongatus UTEX 2973, and Synechocystis sp. PCC 6803.

Aspect 55. The engineered host cell of any one of aspects 33-51, wherein the engineered host cell is a eukaryotic microalgae.

Aspect 56. The engineered host cell of any one of aspects 33-55, wherein the heterologous enzyme comprises a CA polypeptide or functional fragment thereof.

Aspect 57. The engineered host cell of aspect 56, wherein the CA polypeptide is derived from a thermostable CA.

Aspect 58. The engineered host cell of aspect 57, wherein the thermostable CA is from Thermosulfurimonas dismutans.

Aspect 59. The engineered host cell of aspect 58, wherein the CA polypeptide comprises the amino acid sequence of SEQ ID NO: 7.

Aspect 60. The engineered host cell of any one of aspects 33-59, wherein the heterologous nucleic acid is present extrachromosomally.

Aspect 61. The engineered host cell of aspect 60, wherein the heterologous nucleic acid is a plasmid.

Aspect 62. The engineered host cell of any one of aspects 33-59, wherein the heterologous nucleic acid is integrated into the chromosome of the engineered host cell.

Aspect 63. The engineered host cell of aspect 62, wherein the heterologous nucleic acid is integrated into the chromosome by site-specific targeting.

Aspect 64. The engineered host cell of aspect 63, wherein the heterologous nucleic acid is integrated into the chromosome by site-specific targeting using a CRISPR/Cas9 system.

Aspect 65. The engineered host cell of any one of aspects 33-64, wherein the heterologous nucleic acid is operably linked to an inducible promoter.

Aspect 66. A method of producing an engineered OMV comprising:
(a) culturing the engineered host cell according to any one of aspects 33-65 under conditions suitable for expression of the fusion protein; and
(b) obtaining an OMV produced by the engineered host cell, thereby producing the engineered OMV.

Aspect 67. The method of aspect 66, wherein the engineered host cell is cultured in the presence of CO2 and light.

Aspect 68. The method of aspect 66 or 67, further comprising filtering the engineered OMV.

Aspect 69. The method of aspect 68, wherein the filtering is performed by ultrafiltration.

Aspect 70. The method of aspect 68, wherein the filtering is performed by a membrane bioreactor.

Aspect 71. The method of any one of aspects 66-70, further comprising concentrating the engineered OMV.

Aspect 72. A nanocatalyst composition comprising the engineered OMV of any one of aspects 1-31.

Aspect 73. The nanocatalyst composition of aspect 72, wherein the engineered OMV is produced by the method of any one of aspects 66-71.

Aspect 74. The nanocatalyst composition of aspect 72 or 73, comprising the engineered OMV at a concentration greater than about 100 mg/L.

Aspect 75. The nanocatalyst composition of any one of aspects 72-74, comprising the engineered OMV in an amount greater than about 10 g.

Aspect 76. The nanocatalyst composition of aspect 74 or 75, wherein the composition is not a therapeutic composition.

Aspect 77. A nanocatalyst product comprising the nanocatalyst composition of any one of aspects 74-76 in a volume greater than about 1 L.

Aspect 78. A method of reducing CO2 comprising interacting the engineered OMV according to any one of aspects 29-32 with CO2 under a condition suitable for the CA polypeptide or functional fragment thereof to convert CO2 to a catalysis product.

Aspect 79. The method of aspect 78, wherein the catalysis product is a metal carbonate.

Aspect 80. The method of aspect 79, wherein the engineered OMV is interacted with CO₂ in the presence of H₂O and a metal ion under alkaline conditions suitable for (i) the conversion of CO₂ to CO₃² by the CA polypeptide or functional fragment thereof, and (ii) association of CO₃²⁻ with the metal ion to form the metal carbonate.

Aspect 81. The method of aspect 80, wherein the metal ion is Ca2+, Mg2+, Fe2+, Zn2+, Cu2+, Mn2+, or Li+.

Aspect 82. The method of any one of aspects 78-81, wherein the engineered OMV is interacted with CO2 in air, water, or waste.

Aspect 83. The method of any one of aspects 78-82, further comprising producing the engineered OMV according to the method of any one of aspects 66-71.

Aspect 84. The method of aspect 83, wherein producing the engineered OMV is carried out in a first reactor and producing the catalysis product is carried out in a second reactor.

Aspect 85. The method of aspect 84, further comprising isolating engineered OMVs produced in the first reactor and introducing the isolated engineered OMVs into the second reactor.

Aspect 86. The method of aspect 85, wherein isolating the engineered OMVs is carried out by filtration.

Aspect 87. A system for the production of an engineered OMV according to any one of aspects 1-32, the system comprising: (a) a first reactor configured to receive a microorganism capable of producing the engineered OMV: (b) a receptacle configured to receive the engineered OMV produced in the first reactor; and (c) a filtration interface connecting the first reactor to the receptable, wherein the engineered OMV produced in the first reactor is capable of passing through the filtration interface into the receptacle.

Aspect 88. The system of aspect 87, wherein the first reactor is a photobioreactor configured to receive light, and wherein the microorganism is a photosynthetic microorganism.

Aspect 89. The system of aspect 88, wherein the photosynthetic microorganism is a cyanobacterium or a eukaryotic microalga.

Aspect 90. The system of aspect 89, wherein the photosynthetic microorganism is an engineered host cell according to any one of aspects 33-65.

Aspect 91. The system of any one of aspects 87-90, wherein the first reactor is configured to receive ambient air/CO2.

Aspect 92. The system of any one of aspects 87-91, wherein the microorganism in the first reactor is incapable or substantially incapable of passing through the filtration interface into the receptacle.

Aspect 93. The system of any one of aspects 87-92, wherein the filtration interface comprises one or more filtration membranes.

Aspect 94. The system of aspect 93, wherein the filtration interface comprises at least 2, 3, 4, or 5 filtration membranes.

Aspect 95. The system of aspect 93 or 94, wherein the one or more filtration membranes are one or more ultrafiltration membranes.

Aspect 96. The system of any one of aspects 87-95, wherein the receptacle is configured to receive the engineered OMV: (a) at a concentration greater than about 100 mg/L; (b) in an amount greater than about 10 g; and/or (c) in a volume greater than about 100 mL.

Aspect 97. A system for catalyzing the conversion of CO₂ and H₂O to HCO₃₋ and H+, the system comprising: (a) a first reactor configured to receive a microorganism capable of producing an engineered OMV according to any one of aspects 1-32; (b) a second reactor configured to receive the engineered OMV produced in the first reactor in conditions suitable for carrying out the conversion of CO₂ and H₂O to HCO₃₋ and H₊; and (c) a filtration interface connecting the first reactor to the second reactor, wherein the engineered OMV produced in the first reactor is capable of passing through the filtration interface into the second reactor.

Aspect 98. The system of aspect 97, wherein the first reactor is a photobioreactor configured to receive light, and wherein the microorganism is a photosynthetic microorganism.

Aspect 99. The system of aspect 98, wherein the photosynthetic microorganism is a cyanobacterium or a eukaryotic microalga.

Aspect 100. The system of aspect 99, wherein the photosynthetic microorganism is an engineered host cell according to any one of aspects 33-65.

Aspect 101. The system of any one of aspects 97-100, wherein the first reactor is configured to receive ambient air/CO₂.

Aspect 102. The system of any one of aspects 97-101, wherein the second reactor is an alkaline reactor comprising an aqueous composition having a pH greater than about 8.0.

Aspect 103. The system of any one of aspects 97-105, wherein the second reactor is configured to receive CO₂.

Aspect 104. The system of any one of aspects 97-106, wherein the second reactor comprises an aqueous composition comprising a metal ion under conditions suitable for the formation of a metal carbonate comprising the metal ion.

Aspect 105. The system of aspect 107, wherein the metal ion is Ca2+, Mg2+, Fe2+, Zn2+, Cu2+, Mn2+, or Li+.

Aspect 106. The system of any one of aspects 97-108, wherein the microorganism in the first reactor is incapable or substantially incapable of passing through the filtration interface into the second reactor.

Aspect 107. The system of any one of aspects 97-106, wherein the filtration interface comprises one or more filtration membranes.

Aspect 108. The system of aspect 107, wherein the filtration interface comprises at least 2, 3, 4, or 5 filtration membranes.

Aspect 109. The system of aspect 107 or 108, wherein the one or more filtration membranes are one or more ultrafiltration membranes.

Aspect 110. The system of any one of aspects 97-109, wherein the second reactor is configured to receive the engineered OMV: (a) at a concentration greater than about 100 mg/L; (b) in an amount greater than about 10 g; and/or (c) in a volume greater than about 100 mL; (a) at a concentration greater than about 100 mg/L; (b) in an amount greater than about 10 g; and/or (c) in a volume greater than about 100 mL.

### 8. EXAMPLES

### A. Example 1: OMV Engineering

Proof-of-concept experimental studies were performed. They involved expression of fusion proteins in a) E. coli strains (common commercial strains such as BL21 [DE3]) and b) cyanobacterial species (Synechococcus sp. PCC 7942 and UTEX 2973).

Plasmid expression vectors for expressing fusion proteins included pET22b(+), pET25b(+), pBb(RSF1010)1k-GFPuv (Plasmid #106395; Addgene), and pSyn6 (GeneArt Synechococcus Protein Expression Vector). Constructs were conventionally transfected/transformed into cells. Cells were cultured under established conditions. Transfected/transformed cells were isolated using standard techniques.

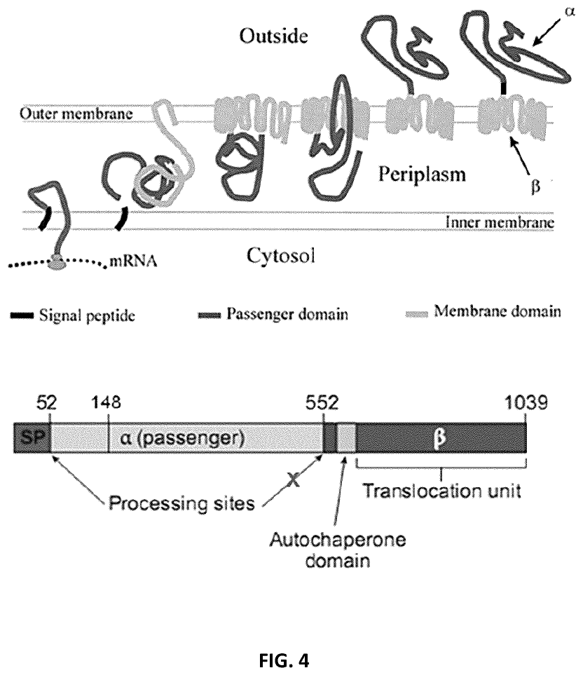

Several fusion protein constructs were evaluated, each of which encodes a thermostable CA from Thermosulfurimonas dismutans (GenBank: OAQ21602.1; Td-CA). The Td-CA was fused, as appropriate, with a signal peptide and a transmembrane protein (e.g., a β-translocator domain from the E. coli autotransporter Ag43 as shown in FIG. 4). The constructs generated are summarized in Table 1.

TABLE 1

| | | | SEQ ID NO |
|---|---|---|---|
| Construct/Host cell | Fusion protein (FP) insert | Vector | (FP) |
| Ag43SP-TdCA-Ag43β- | TdCA-Ag43 fusion protein: | pET25b(+) | 1 |
| pET25b(+)/ | Ag 43 E. coli signal peptide + | | |
| Escherichia coli K-12 | Td carbonic anhydrase/CA + | | |
| | linker + Ag43 β-chain | | |
| | translocator domain | | |
| 6xHis-TdCA-Ag43- | TdCA-Ag43 fusion protein | pET25b(+) | |
| pET25b(+)/ | with 6X His | | |
| Escherichia coli K-12 | | | |
| SynSP-TdCA-Ag43β-pSyn6/ | TdCA-Ag43 fusion protein: | pSyn6 | 2 |
| Synechococcus elongatus | Synechococcus PCC 7942 27- | | |
| PCC 7942 or UTEX 2973 | AA signal peptide + Td | | |
| | carbonic anhydrase/CA + | | |
| | linker + Ag43 β-chain | | |
| | translocator domain | | |
| 6xHis-TdCA-Ag43- | TdCA-Ag43 fusion protein | pBb(RSF1010)1k- | |
| pBb(RSF1010)1k-GFPuv/ | with 6X His | GFPuv | |
| Synechocystis sp. | | | |
| (ATCC 27184) | | | |
| 6xHis-TdCA-Ag43-pSyn6/ | TdCA-Ag43 fusion protein | pSyn6 | |
| Synechococcus elongatus | with 6X His | | |
| PCC 7942 | | | |
| SP-TdCA-tHbp-pET25b(+)/ | TdCA-Hbp fusion protein: | pET25b(+) | 3 |
| Escherichia coli K-12 | signal peptide (Daleke- | | |
| | Schermerhorn et al.) + Td | | |
| | carbonic anhydrase/CA + | | |
| | linker + truncated Hbp | | |
| | passenger domain: minus the | | |
| | N-terminal D1 subdomain + | | |
| | the remaining subdomains + | | |
| | the β domain | | |
| tSomA-TdCA-pSyn6/ | SomA-TdCA fusion protein: | pSyn6 | 4 |
| Synechococcus elongatus | Truncated Synechococcus | | |
| PCC 7942 or UTEX 2973 | SomA with the C-terminus at | | |
| | amino acid position 382 | | |
| | (located at the fifth cell- | | |
| | surface exposed loop) + Td | | |
| | carbonic anhydrase/CA | | |
| tMipA-TdCA-pET25b(+)/ | MipA-TdCA fusion protein: | pET25b(+) | 5 |
| Escherichia coli K-12 | truncated MipA with the C- | | |
| | terminus at amino acid position | | |
| | 140 located at the third cell- | | |
| | surface exposed loop + linker + | | |
| | Td carbonic anhydrase/CA | | |
| pelBSP-INPN- | INPN-RE-TdCA fusion | pET22b(+) | 6 |
| TdCA-pET22b(+)/ | protein: pelB signal | | |
| Escherichia coli K-12 | peptide + INPN domain from | | |
| | Pseudomonas syringae + | | |
| | linker + Td carbonic | | |
| | anhydrase/CA + His-Tag | | |

Fusion protein expression was evaluated by colony RT-PCR at the mRNA level and by immunoblotting at the protein level in both total cell lysates and lysates of OMVs. OMVs were obtained from cell culture supernatants by polyethylene glycol (PEG) precipitation and evidence of TdCA-Ag43 fusion protein expression in the OMVs was detected by immunoblotting.

Purified engineered OMV preparations were tested and found to have CA activity above background using standard techniques.

The study demonstrated successful expression of the fusion protein and enzyme activity in OMVs obtained from the engineered bacteria.

### B. Example 2: Membrane Accumulation of TdCA-Ag43 Protein in UTEX 2973 Cells

Carbonic anhydrase (CA) that can function at high temperatures (over 60° C.) from the bacterium Thermosulfurimonas dismutans (Td) was selected. TdCA was fused with a truncated E. coli autotransporter called antigen 43 (Ag43; FIG. 1; SEQ ID NO: 1). The coding sequence of the construct was inserted into a Synechococcus-specific plasmid vector with a strong, constitutive (continuous transcription) promoter at the NdeI restriction site (pSyn6; Thermo Fischer). The resulting recombinant plasmid (pSyn6-TdCA-Ag43; SEQ ID NO: 24) was transformed into the fast-growing cyanobacterium Synechococcus elongatus UTEX 2973 using tri-conjugal transformation, aided by one population of E. coli HB101 transformed with plasmid pRL623 and the pSyn6 plasmid and another population of E. coli HB101 transformed with plasmid pRL443. Following antibiotic selection by 10 μg/mL spectinomycin resistance on BG-11 (Millipore Sigma) agar plates, colonies were transferred to liquid BG-11 media (Millipore Sigma) supplemented with 10 μg/mL spectinomycin and incubated at 37° C. in 2.5% CO₂ under a light intensity of 60 μmol m⁻² s⁻¹ with shaking.

To detect expression of the fusion protein TdCA-Ag43 in transformed cyanobacteria, whole cells were lysed by vortexing with glass beads. To suppress proteolysis, complete protease inhibitor (MilliporeSigma) was added to the lysis mixture prior to lysis. Next, samples were mixed with 2× Laemmli buffer (BioRAD) and heated to 95° C. for 5 minutes. After centrifugation, samples were loaded on and electrophoresed through a 4-20% gradient SDS-PAGE gel (BioRAD). Protein was then transferred to a PVDF membrane (ImmobilonP; MilliporeSigma). The membrane was blocked with 5% bovine serum albumin in Tris-buffered saline for 20 minutes and incubated with a monoclonal anti-TdCA antibody (GenScript, USA).

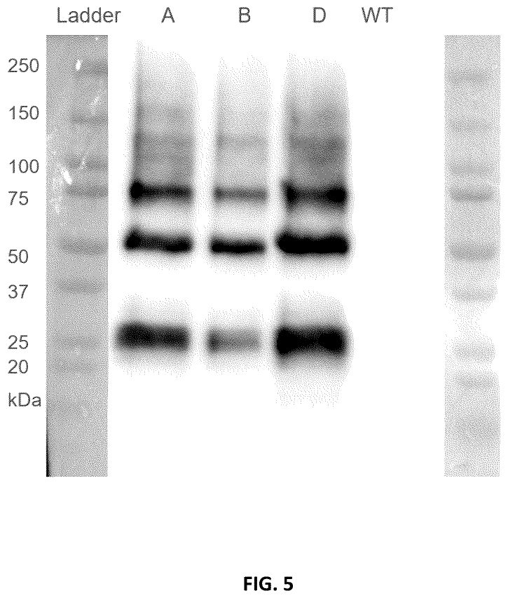

After 5 washes, the membrane was incubated for 1 hour with a secondary, HRP-conjugated antibody (Abcam, USA), washed, and then chemiluminesence was developed using Clarity substrate (BioRAD) and visualized using a ChemiDocMP+ imaging system (BioRAD). It was confirmed that the correct fusion protein was expressed with the expected molecular weight (FIG. 5: ˜78 kDa). Smaller bands were also observed with the sizes indicating cleavage of the fusion protein (TdCA about 28 kDa and Ag43 about 52 kDa).

### Results

FIG. 5 shows immunoblot of samples A, B, and D, separate UTEX 2973 colonies stably transfected with TdCA-Ag43 (pSyn_6 (constitutive expression vector). Wild type (WT) included untransfected cells. The presence of a band at 78 kDa confirmed the expression of TdCA-Ag43 in the transformed cells (FIG. 5). The presence of additional bands (FIG. 5: <78 kDa) was related to cleavage fragments of the fusion protein containing the TdC Antigen.

### C. Example 3: Evidence of Whole Cell Cyanobacterial Cell Carbonic Anhydrase (CA) Activity Involving Whole UTEX 2973 Cells and OMVs

Overall carbonic anhydrase (CA) activity of whole cyanobacterial cells was evaluated in whole UTEX 2973 cells and OMVs.

### Methods:

A colorimetric assay was used to monitor the rate of pH change caused by CA activity using phenol red as an indicator.

In step 1, whole cell samples were washed 2× with growth media, then washed 2× and resuspended in pH 8.5, 20 mM Tris-HCl. In step 2, cell density was measured optically at 750 nm (OD₇₅₀).

In step 3, in a quartz cuvette, 600 μL of a pH 8.5, 20 mM Tris-HCl and 1 mM phenol red solution was combined with a 10 μL sub-sample of the concentrated cells and 400 μL ice-cold water supersaturated with CO₂.

In step 4, while stirring, changes in absorbance at 570 nm over time were recorded using an Agilent Cary 3500 UV-Vis System cooled to 4° C. In step 5, the Wilbur-Anderson activity (WA) was calculated using the following:

$$WA = ( {t}_{0} - t ) / t$$

where t, is the time taken for the concentrated cell sample to record a pH change from 7.5 to 6.5 (corresponding to absorbency values of 1.2 and 0.18, respectively) and to is time taken for the blank (20 mM Tris-HCl) to record any pH change from 7.5 to 6.5.

In step 6, WA was divided by the OD₇₅₀ of the sample obtained in step 2, to account for variation in cell density across samples, and by 0.001 mL to obtain a per-volume measurement.

### Results

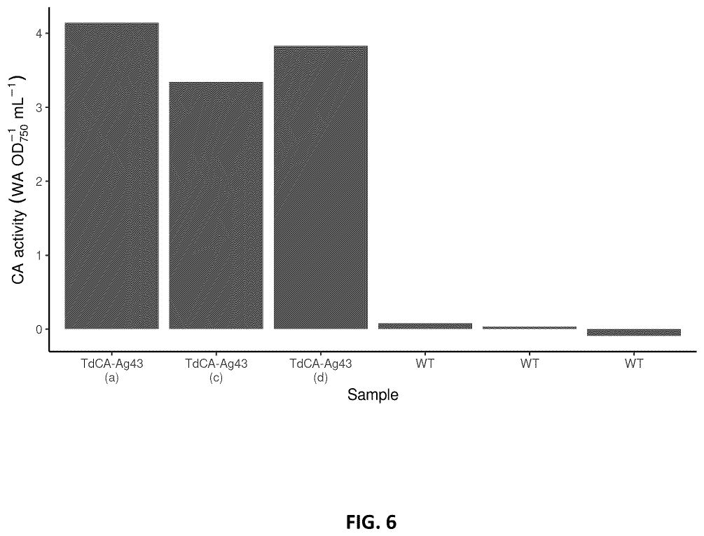

The results as shown in FIG. 6 indicated significantly more whole cell CA activity in cells transformed with the TdCA-Ag43 construct as described in Example 2 compared to wild type cells.

### D. Example 4: Proteinase K Evidence for Cell Surface Accumulation of TdCA-Ag43

Proteinase K digestion of cyanobacterial surface-displayed proteins was assessed.

Proteinase K is a cysteine protease that is impermeable to a whole/intact bacterial cell. To detect cell surface display of the TdCA protein (SEQ ID NO: 1), a proteinase K digestion of protein on the outside of whole cells was performed.

For each culture, six 1 mL samples were collected, centrifuged at 6000×g and re-suspended in phosphate buffer (pH 7.4). Next, 0, 0.1, 0.2, 0.5, 1.0, or 1.5 U proteinase K (Ferri et al. 2015) were added to each of the six samples and incubated at 37° C. for 30 minutes. The cells were then washed with PBS (pH 7.4) and an immunoblot was performed with the process as described in Example 2.

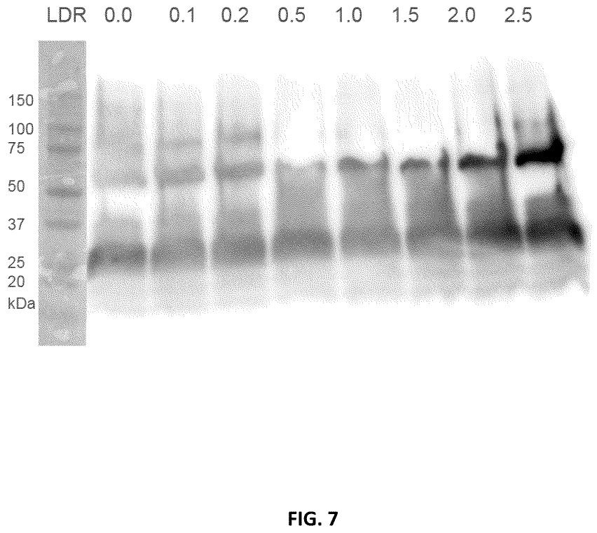

A replicate polyacrylamide gel was stained with Comassie blue as a control for total protein loading and visualized using the Gel Doc XR+ system (BioRad). Evidence was found of surface protein digestion at high proteinase K concentrations (esp. 1 U) inTdCA-Ag43 culture D. To confirm evidence of proteinase K digestion in TdCA-Ag43 replicate culture D, a follow-up proteinase K assay was performed, as described above, but with eight samples, and addition of 0, 0.1, 0.2, 0.5, 1.0, 1.5, 2.0, or 2.5 U proteinase K, as shown in FIG. 7.

### Results

In the treatments of whole cell samples of clonal culture D with different concentrations of proteinase K, as shown in FIG. 7, the immunoblotting band near 78 kDa, which corresponds to the expected size of the TdCA-Ag43 fusion protein, showed significant decrease in the intensity with increasing proteinase K concentrations.

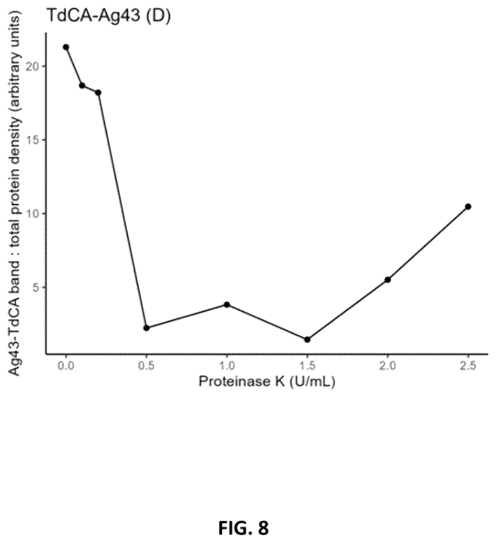

FIG. 8 provides quantitative analysis (measuring band volume/density) of the results in FIG. 7. These results suggest cell surface accumulation of TdCA-Ag43 (immunoblot for sample D; Units proteinase K/mL) since proteinase K works mostly on the cell surface.

### E. Example 5: CA Activity of OMV from UTEX 2973 Cell Supernatants

This study assessed carbonic anhydrase activity of outer membrane vesicles/OMVs in UTEX 2973 cell supernatants.

### Methods

OMVs were isolated from ˜40 mL samples of cell culture with an OD750>1. The culture samples were centrifuged at 6000×g and supernatants were filtered using 0.22 μm syringe filters. Next, the filtered supernatants were spin-concentrated to 250-1000 μL using 50 MWCO filter units (Amicon). An ExoQuick-TC kit (SBI) was then used to pellet OMVs and re-suspend them in pH 7.4 phosphate buffer.

Total protein concentration in the samples was estimated using a microvolume spectrometer (NanoDrop). Dynamic light scattering was used to confirm that the size distribution of particles in the sample corresponds to the expected size of OMVs (e.g., 100-200 nm). CA activity assays were performed as detailed for Example 3. In this case, no normalization was performed on the measurements. The experiment was repeated four times, and the data were used to fit a linear mixed model, where, the sample type (WT, A, C, D) was the predictor, and the date of the experiment was used as a random intercept to control for variations across the different replicate experiments.

### Results

An analysis of variance was performed on the data. The results showed that there was a significant effect of sample type on the CA activity (ANOVA: F3,12=6.28; p=0.0083).

A Tukey-corrected pairwise post-hoc test indicated a significant difference of 0.24±0.07 WA units (±SE: p=0.011) between the D and wild type sample types.

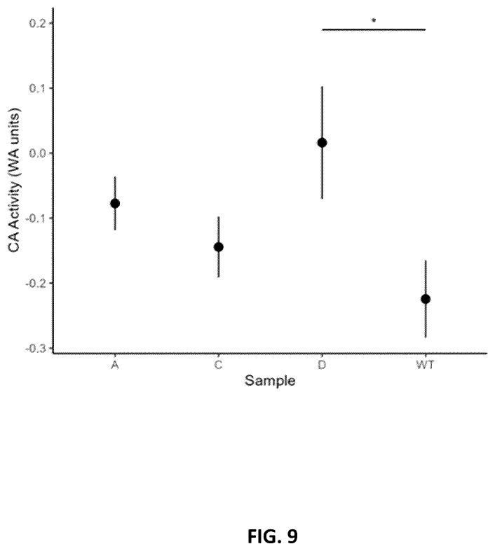

As shown in FIG. 9, there was a statistically significant level of carbonic anhydrase activity on OMVs isolated from at least one of the three cyanobacterial cells expressing the Ag43-TdCA fusion protein (SEQ ID NO: 1).

### F. Example 6: Liquid Chromatography-Mass Spectrometry (LC-MS) Study of SomA-TdCA Expression in UTEX 2973 Cells

TdCA was fused with a Synechococcus outer membrane protein A, somatostatin A (somA; SEQ ID NO: 4). The coding sequence of the construct was inserted into a Synechococcus-specific plasmid vector with a strong, constitutive (continuous transcription) promoter at the NdeI restriction site (pSyn6; Thermo Fischer). The resulting recombinant plasmid (tSomA-TdCA-pSyn6; SEQ ID NO: 4) was transformed into the fast-growing cyanobacterium Synechococcus elongatus UTEX 2973 cells. As shown by Mass Spectrometry, the full length of the tSOMA-TdCA fusion protein is 67,113.57 Da.

To detect expression of the fusion protein tSomA-TdCA in transformed cyanobacteria, whole cells were lysed by vortexing with glass beads. To suppress proteolysis, complete protease inhibitor (MilliporeSigma) was added to the lysis mixture prior to lysis. Next, samples were mixed with 2× Laemmli buffer (BioRAD) and heated to 95° C. for 5 minutes. After centrifugation, samples were loaded on and electrophoresed through a 4-20% gradient SDS-PAGE gel (BioRAD). Protein was then transferred to a PVDF membrane (ImmobilonP; MilliporeSigma). The membrane was blocked with 5% bovine serum albumin in Tris-buffered saline for 20 minutes and incubated with a monoclonal anti-TdCA antibody (GenScript, USA).

After 5 washes, the membrane was incubated for 1 hour with a secondary, HRP-conjugated antibody (Abcam, USA), washed, and then chemiluminesence was developed using Clarity substrate (BioRAD) and visualized using a ChemiDocMP+ imaging system (BioRAD).

The experiment confirmed expression of the fusion protein although some of the fusion protein was cleaved, resulting in bands with smaller sizes.

### 9. EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents, and patent applications cited within the body of the instant specification are hereby incorporated by reference in their entirety, for all purposes.

This application claims the benefit of U.S. Provisional Patent Application No. 63/653,187 filed on May 29, 2024, the entire contents of which are hereby incorporated by reference.

SEQUENCES

| SEQ | | |
|---|---|---|
| ID | | |
| NO | Description | Sequence |
| 1 | Thermosulfurimonas | MKRHLNTCYRLVWNHMTGAFVVASELARARGKRGGVAVALSLAAVTSLPVLAGGGHV |
| | dismutans | VKWGYVGKIGPAHWGDLAHEYFMCKVGKNQSPVDINSSVTIEAQLEPINFHYRDQISGE |
| | (TdCA); Escherichia | IVNNGHTIMVVPKEDNYIVVDGKKFHLKQFHFHSPSEHTVEGKYYLLELHFVHQADDGQ |
| | coli (strain K12), | LAVIGVVFDRGAEHPEIAKLWKEAPEHEGKKELKSLVNMQALLPENLDYYRYSGSLTTPP |
| | Ag43 beta-barrel | CSEGVIWLFLKNPLQISEAQAEKFKKIMGFENNRPVQPVNARKILKGSSSGSSPTNVTLAS |
| | domain | GATWNIPDNATVQSVVDDLSHAGQIHFTSTRTGKFVPATLKVKNLNGQNGTISLRVRPD |
| | TdCA-Ag43 fusion | MAQNNADRLVIDGGRATGKTILNLVNAGNSASGLATSGKGIQVVEAINGATTEEGAFV |
| | protein: Ag 43 E. | QGNRLQAGAFNYSLNRDSDESWYLRSENAYRAEVPLYASMLTQAMDYDRIVAGSRSH |
| | coli signal peptide | QTGVNGENNSVRLSIQGGHLGHDNNGGIARGATPESSGSYGFVRLEGDLMRTEVAGM |
| | (amino acid 1-52) + | SVTAGVYGAAGHSSVDVKDDDGSRAGTVRDDAGSLGGYLNLVHTSSGLWADIVAQGT |
| | Td carbonic | RHSMKASSDNNDFRARGWGWLGSLETGLPFSITDNLMLEPQLQYTWQGLSLDDGKDN |
| | anhydrase/CA | AGYVKFGHGSAQHVRAGFRLGSHNDMTFGEGTSSRAPLRDSAKHSVSELPVNWWVQ |
| | (amino acids 53- | PSVIRTFSSRGDMRVGTSTAGSGMTFSPSQNGTSLDLQAGLEARVRENITLGVQAGYAH |
| | 281) + linker | SVSGSSAEGYNGQATLNVTF |
| | (amino acids 282- | |
| | 288) + Ag43 β- | |
| | chain translocator | |
| | domain (amino | |
| | acids 289-776): | |
| 2 | TdCA-Ag43 fusion | MKRLFSALLLAPAIAGVAAGAANANGLGGGHVVKWGYVGKIGPAHWGDLAHEYFMC |
| | protein: | KVGKNQSPVDINSSVTIEAQLEPINFHYRDQISGEIVNNGHTIMVVPKEDNYIVVDGKKF |
| | Synechococcus PCC | HLKQFHFHSPSEHTVEGKYYLLELHFVHQADDGQLAVIGVVFDRGAEHPEIAKLWKEAP |
| | 7942 27-AA signal | EHEGKKELKSLVNMQALLPENLDYYRYSGSLTTPPCSEGVIWLFLKNPLQISEAQAEKFKKI |
| | peptide (amino | MGFENNRPVQPVNARKILKGSSSGSSPTNVTLASGATWNIPDNATVQSVVDDLSHAGQ |
| | acids 1-27) + Td | IHFTSTRTGKFVPATLKVKNLNGQNGTISLRVRPDMAQNNADRLVIDGGRATGKTILNLV |
| | carbonic | NAGNSASGLATSGKGIQVVEAINGATTEEGAFVQGNRLQAGAFNYSLNRDSDESWYLR |
| | anhydrase/CA | SENAYRAEVPLYASMLTQAMDYDRIVAGSRSHQTGVNGENNSVRLSIQGGHLGHDNN |
| | (amino acids 28- | GGIARGATPESSGSYGFVRLEGDLMRTEVAGMSVTAGVYGAAGHSSVDVKDDDGSRA |
| | 256) + linker | GTVRDDAGSLGGYLNLVHTSSGLWADIVAQGTRHSMKASSDNNDFRARGWGWLGSL |
| | (amino acids 257- | ETGLPFSITDNLMLEPQLQYTWQGLSLDDGKDNAGYVKFGHGSAQHVRAGFRLGSHN |
| | 263) + Ag43 β- | DMTFGEGTSSRAPLRDSAKHSVSELPVNWWVQPSVIRTFSSRGDMRVGTSTAGSGMT |
| | chain translocator | FSPSQNGTSLDLQAGLEARVRENITLGVQAGYAHSVSGSSAEGYNGQATLNVTF |
| | domain (amino | |
| | acids 264-751) | |
| 3 | TdCA-Hbp fusion | MNRIYSLRYSAVARGFIAVSEFARKCVHKSVRRLCFPVLLLIPVLFSAGSLAGGGGHVVKW |
| | protein: signal | GYVGKIGPAHWGDLAHEYFMCKVGKNQSPVDINSSVTIEAQLEPINFHYRDQISGEIVN |
| | peptide (Daleke- | NGHTIMVVPKEDNYIVVDGKKFHLKQFHFHSPSEHTVEGKYYLLELHFVHQADDGQLAV |
| | Schermerhorn et | IGVVFDRGAEHPEIAKLWKEAPEHEGKKELKSLVNMQALLPENLDYYRYSGSLTTPPCSE |
| | al.) (amino acids 1- | GVIWLFLKNPLQISEAQAEKFKKIMGFENNRPVQPVNARKILKGSSSGSSNDAPVTFRTS |
| | 53) + Td carbonic | EGGALEWSFNSSTGAGALTQGTTTYAMHGQQGNDLNAGKNLIFQGQNGQINLKDSVS |
| | anhydrase/CA | QGAGSLTFRDNYTVTTSNGSTWTGAGIVVDNGVSVNWQVNGVKGDNLHKIGEGTLTV |
| | (amino acids 54- | QGTGINEGGLKVGDGKVVLNQQADNKGQVQAFSSVNIASGRPTVVLTDERQVNPDTV |
| | 282) + linker | SWGYRGGTLDVNGNSLTFHQLKAADYGAVLANNVDKRATITLDYALRADKVALNGWS |
| | (amino acids 283- | ESGKGTAGNLYKYNNPYTNTTDYFILKQSTYGYFPTDQSSNATWEFVGHSQGDAQKLVA |
| | 289) + truncated | DRFNTAGYLFHGQLKGNLNVDNRLPEGVTGALVMDGAADISGTFTQENGRLTLQGHP |
| | Hbp passenger | VIHAYNTQSVADKLAASGDHSVLTQPTSFSQEDWENRSFTFDRLSLKNTDFGLGRNATL |
| | domain: minus the | NTTIQADNSSVTLGDSRVFIDKNDGQGTAFTLEEGTSVATKDADKSVFNGTVNLDNQSV |
| | N-terminal D1 | LNINDIFNGGIQANNSTVNISSDSAVLGNSTLTSTALNLNKGANALASQSFVSDGPVNISD |
| | subdomain + the | ATLSLNSRPDEVSHTLLPVYDYAGSWNLKGDDARLNVGPYSMLSGNINVQDKGTVTLG |
| | remaining | GEGELSPDLTLQNQMLYSLFNGYRNIWSGSLNAPDATVSMTDTQWSMNGNSTAGNM |
| | subdomains + the b | KLNRTIVGFNGGTSPFTTLTTDNLDAVQSAFVMRTDLNKADKLVINKSATGHDNSIWVN |
| | domain (amino acids | FLKKPSNKDTLDIPLVSAPEATADNLFRASTRVVGFSDVTPILSVRKEDGKKEWVLDGYQV |
| | 290-1359) | ARNDGQGKAAATFMHISYNNFITEVGSLNKRMGDLRDINGEAGTWVRLLNGSGSADG |
| | | GFTDHYTLLQMGADRKHELGSMDLFTGVMATYTDTDASADLYSGKTKSWGGGFYASG |
| | | LFRSGAYFDVIAKYIHNENKYDLNFAGAGKQNFRSHSLYAGAEVGYRYHLTDTTFVEPQA |
| | | ELVWGRLQGQTFNWNDSGMDVSMRRNSVNPLVGRTGVVSGKTFSGKDWSLTARAG |
| | | LHYEFDLTDSADVHLKDAAGEHQINGRKDSRMLYGVGLNARFGDNTRLGLEVERSAFGK |
| | | YNTDDAINANIRYSF |
| 4 | SomA-TdCA fusion | MKRLFSALLLAPAIAGVAAGAANANGLSTEQLQKIDAVTPNGITSGQITSITELSDVKPTD |
| | protein: Truncated | WAYQALQSLVERYGCIVGYPDRTYRGSRPLSRYEFAAGLNACLDKVIEFAASKEDLDTLKR |
| | Synechococcus SomA | LTEEFQAELATLRGRVDSLEARVKELEATRFSTTTKLQGEVIFSLDAVANTAGNERNQDG |
| | with the C- | AVSFGNRVSLNLNTSFTGKDLLLTRLRARNIETIQQRLSPGFNPSGSRLDYDGTGSPGVPN |
| | terminus at amino | SANTFFLDKLLYRFPVGDVSFTVGTAGVQPQDYGLSDATFFSGPANTKAFKYVGAGVYA |
| | acid position 382 | DTRDADTAGVGFNWKASKNFSFQAGYINRNSADVSTVNSGGVFGFTPTGTGTNSWDV |
| | (located at the | NAQVKYQTDNNKFRVALAYALRNGGGHVVKWGYVGKIGPAHWGDLAHEYFMCKVG |
| | fifth cell-surface | KNQSPVDINSSVTIEAQLEPINFHYRDQISGEIVNNGHTIMVVPKEDNYIVVDGKKFHLKQ |
| | exposed loop) | FHFHSPSEHTVEGKYYLLELHFVHQADDGQLAVIGVVFDRGAEHPEIAKLWKEAPEHEG |
| | (amino acids 1-382) | KKELKSLVNMQALLPENLDYYRYSGSLTTPPCSEGVIWLFLKNPLQISEAQAEKFKKIMGF |
| | + Td carbonic | ENNRPVQPVNARKILK |
| | anhydrase/CA | |
| | (amino acids 383- | |
| | 613) | |
| 5 | MipA-TdCA fusion | MTKLKLLALGVLIATSAGVAHAEGKFSLGAGVGVVEHPYKDYDTDVYPVPVINYEGDNF |
| | protein: truncated | WFRGLGGGYYLWNDATDKLSITAYWSPLYFKAKDSGDHQMRHLDDRKSTMMAGLSY |
| | MipA with the C- | AHFTQYGYLRTTLAGDTLDNSNGIV GGGGSGGSGGGGSGGGGSGGGGS |
| | terminus at amino | GGGHVVKWGYVGKIGPAHWGDLAHEYFMCKVGKNQSPVDINSSVTIEAQLEPINFH |
| | acid position 140 | YRDQISGEIVNNGHTIMVVPKEDNYIVVDGKKFHLKQFHFHSPSEHTVEGKYYLLELHF |
| | located at the third | VHQADDGQLAVIGVVFDRGAEHPEIAKLWKEAPEHEGKKELKSLVNMQALLPENLDY |
| | cell-surface exposed | YRYSGSLTTPPCSEGVIWLFLKNPLQISEAQAEKFKKIMGFENNRPVQPVNARKILK |
| | loop (amino | |
| | acids 1-140) + | |
| | linker (amino | |
| | acids 141-163) + Td | |
| | carbonic | |
| | anhydrase/CA | |
| | (amino acids 164-392) | |
| 6 | INPN-RE-TdCA | MKYLLPTAAAGLLLLAAQPAMAMTLDKALVLRTCANNMADHCGLIWPASGTVESRYW |
| | fusion protein: | QSTRRHENGLVGLLWGAGTSAFLSVHADARWIVCEVAVADIISLEEPGMVKFPRAEVVH |
| | pelB signal peptide | VGDRISASHFISARQADPASTSTSTSTSTLTPMPTAIPTPMPAVASVTLPVAEQARHEVFD |
| | (amino acids 1-22) | VASVSAAAAPVNTLPVTTPQNLQTATYGSTLSGDNHSRLIAGYGSNETAGNHSDLIGGG |
| | + INPN (: | GSGGGHVVKWGYVGKIGPAHWGDLAHEYFMCKVGKNQSPVDINSSVTIEAQLEPINFH |
| | underlining: front | YRDQISGEIVNNGHTIMVVPKEDNYIVVDGKKFHLKQFHFHSPSEHTVEGKYYLLELHFV |
| | two sub-repeats in | HQADDGQLAVIGVVFDRGAEHPEIAKLWKEAPEHEGKKELKSLVNMQALLPENLDYYRY |
| | the middle repeat | SGSLTTPPCSEGVIWLFLKNPLQISEAQAEKFKKIMGFENNRPVQPVNARKILKHHHHHH |
| | domain of ice | |
| | nucleoprotein) | |
| | (amino acids 23- | |
| | 233)+ linker (amino | |
| | acids 234-238) + Td | |
| | carbonic | |
| | anhydrase/CA | |
| | (amino acids 239- | |
| | 467)+ His-Tag | |
| | (amino acids 468-473) | |
| 7 | Td carbonic | GGGHVVKWGYVGKIGPAHWGDLAHEYFMCKVGKNQSPVDINSSVTIEAQLEPINFHYR |
| | anhydrase | DQISGEIVNNGHTIMVVPKEDNYIVVDGKKFHLKQFHFHSPSEHTVEGKYYLLELHFVHQ |
| | | ADDGQLAVIGVVFDRGAEHPEIAKLWKEAPEHEGKKELKSLVNMQALLPENLDYYRYSG |
| | | SLTTPPCSEGVIWLFLKNPLQISEAQAEKFKKIMGFENNRPVQPVNARKILK |
| 8 | Ag43 b-chain | PTNVTLASGATWNIPDNATVQSVVDDLSHAGQIHFTSTRTGKFVPATLKVKNLNGQNG |
| | translocator | TISLRVRPDMAQNNADRLVIDGGRATGKTILNLVNAGNSASGLATSGKGIQVVEAINGA |
| | domain | TTEEGAFVQGNRLQAGAFNYSLNRDSDESWYLRSENAYRAEVPLYASMLTQAMDYDRI |
| | | VAGSRSHQTGVNGENNSVRLSIQGGHLGHDNNGGIARGATPESSGSYGFVRLEGDLMR |
| | | TEVAGMSVTAGVYGAAGHSSVDVKDDDGSRAGTVRDDAGSLGGYLNLVHTSSGLWA |
| | | DIVAQGTRHSMKASSDNNDFRARGWGWLGSLETGLPFSITDNLMLEPQLQYTWQGLS |
| | | LDDGKDNAGYVKFGHGSAQHVRAGFRLGSHNDMTFGEGTSSRAPLRDSAKHSVSELPV |
| | | NWWVQPSVIRTFSSRGDMRVGTSTAGSGMTFSPSQNGTSLDLQAGLEARVRENITLGV |
| | | QAGYAHSVSGSSAEGYNGQATLNVTF |
| 9 | Truncated Hbp | NDAPVTFRTSEGGALEWSFNSSTGAGALTQGTTTYAMHGQQGNDLNAGKNLIFQGQN |
| | passenger domain: | GQINLKDSVSQGAGSLTFRDNYTVTTSNGSTWTGAGIVVDNGVSVNWQVNGVKGDNL |
| | minus the N- | HKIGEGTLTVQGTGINEGGLKVGDGKVVLNQQADNKGQVQAFSSVNIASGRPTVVLTD |
| | terminal D1 | ERQVNPDTVSWGYRGGTLDVNGNSLTFHQLKAADYGAVLANNVDKRATITLDYALRAD |
| | subdomain + the | KVALNGWSESGKGTAGNLYKYNNPYTNTTDYFILKQSTYGYFPTDQSSNATWEFVGHS |
| | remaining | QGDAQKLVADRFNTAGYLFHGQLKGNLNVDNRLPEGVTGALVMDGAADISGTFTQEN |
| | subdomains + the b | GRLTLQGHPVIHAYNTQSVADKLAASGDHSVLTQPTSFSQEDWENRSFTFDRLSLKNTD |
| | domain | FGLGRNATLNTTIQADNSSVTLGDSRVFIDKNDGQGTAFTLEEGTSVATKDADKSVFNG |
| | | TVNLDNQSVLNINDIFNGGIQANNSTVNISSDSAVLGNSTLTSTALNLNKGANALASQSF |
| | | VSDGPVNISDATLSLNSRPDEVSHTLLPVYDYAGSWNLKGDDARLNVGPYSMLSGNINV |
| | | QDKGTVTLGGEGELSPDLTLQNQMLYSLFNGYRNIWSGSLNAPDATVSMTDTQWSM |
| | | NGNSTAGNMKLNRTIVGFNGGTSPFTTLTTDNLDAVQSAFVMRTDLNKADKLVINKSA |
| | | TGHDNSIWVNFLKKPSNKDTLDIPLVSAPEATADNLFRASTRVVGFSDVTPILSVRKEDGK |
| | | KEWVLDGYQVARNDGQGKAAATFMHISYNNFITEVGSLNKRMGDLRDINGEAGTWV |
| | | RLLNGSGSADGGFTDHYTLLQMGADRKHELGSMDLFTGVMATYTDTDASADLYSGKTK |
| | | SWGGGFYASGLFRSGAYFDVIAKYIHNENKYDLNFAGAGKQNFRSHSLYAGAEVGYRYH |
| | | LTDTTFVEPQAELVWGRLQGQTFNWNDSGMDVSMRRNSVNPLVGRTGVVSGKTFSG |
| | | KDWSLTARAGLHYEFDLTDSADVHLKDAAGEHQINGRKDSRMLYGVGLNARFGDNTRL |
| | | GLEVERSAFGKYNTDDAINANIRYSF |
| 10 | Truncated | MKRLFSALLLAPAIAGVAAGAANANGLSTEQLQKIDAVTPNGITSGQITSITELSDVKPTD |
| | Synechococcus | WAYQALQSLVERYGCIVGYPDRTYRGSRPLSRYEFAAGLNACLDKVIEFAASKEDLDTLKR |
| | SomA with the C- | LTEEFQAELATLRGRVDSLEARVKELEATRFSTTTKLQGEVIFSLDAVANTAGNERNQDG |
| | terminus at amino | AVSFGNRVSLNLNTSFTGKDLLLTRLRARNIETIQQRLSPGFNPSGSRLDYDGTGSPGVPN |
| | acid position 382 | SANTFFLDKLLYRFPVGDVSFTVGTAGVQPQDYGLSDATFFSGPANTKAFKYVGAGVYA |
| | | DTRDADTAGVGFNWKASKNFSFQAGYINRNSADVSTVNSGGVFGFTPTGTGTNSWDV |
| | | NAQVKYQTDNNKFRVALAYALRN |
| 11 | Truncated MipA | MTKLKLLALGVLIATSAGVAHAEGKFSLGAGVGVVEHPYKDYDTDVYPVPVINYEGDNF |
| | with the C- | WFRGLGGGYYLWNDATDKLSITAYWSPLYFKAKDSGDHQMRHLDDRKSTMMAGLSY |
| | terminus at amino | AHFTQYGYLRTTLAGDTLDNSNGIV |
| | acid position 140 | |
| | located at the third | |
| | cell-surface | |
| | exposed loop | |
| 12 | INPN from | MTLDKALVLRTCANNMADHCGLIWPASGTVESRYWQSTRRHENGLVGLLWGAGTSAF |
| | Pseudomonas | LSVHADARWIVCEVAVADIISLEEPGMVKFPRAEVVHVGDRISASHFISARQADPASTSTS |
| | syringae | TSTSTLTPMPTAIPTPMPAVASVTLPVAEQARHEVFDVASVSAAAAPVNTLPVTTPQNL |
| | | QTATYGSTLSGDNHSRLIAGYGSNETAGNHSDLI |
| 13 | Ag 43 E. coli signal | MKRHLNTCYRLVWNHMTGAFVVASELARARGKRGGVAVALSLAAVTSLPVLA |
| | peptide | |
| 14 | Synechococcus PCC | MKRLFSALLLAPAIAGVAAGAANANGL |
| | 7942 27-AA signal | |
| | peptide | |
| 15 | pelB signal peptide | MKYLLPTAAAGLLLLAAQPAMA |
| 16 | signal peptide | MNRIYSLRYSAVARGFIAVSEFARKCVHKSVRRLCFPVLLLIPVLFSAGSLAG |
| 17 | Linker | GSSSGSS |
| 18 | G4S linker | GGGGS |
| 19 | G4SGGS(G4S)3 | GGGGSGGSGGGGSGGGGSGGGGS |
| | linker | |
| 20 | sp\|P39180\|AG43_ | |
| | ECOLI Antigen 43 | |
| | OS = Escherichia coli | MKRHLNTCYRLVWNHMTGAFVVASELARARGKRGGVAVALSL |
| | (strain K12) | AAVTSLPVLAADIVVHPGETVNGGTLANHDNQIVFGTTNGMT |
| | OX = 83333 GN = flu | ISTGLEYGPDNEANTGGQWVQDGGTANKTTVTSGGLQRVNPG |
| | PE = 1 SV = 3 | GSVSDTVISAGGGQSLQGRAVNTTLNGGEQWMHEGAIATGTV |
| | | INDKGWQVVKPGTVATDTVVNTGAEGGPDAENGDTGQFVRGD |
| | | AVRTTINKNGRQIVRAEGTANTTVVYAGGDQTVHGHALDTTL |
| | | NGGYQYVHNGGTASDTVVNSDGWQIVKNGGVAGNTTVNQKGR |
| | | LQVDAGGTATNVTLKQGGALVTSTAATVTGINRLGAFSVVEG |
| | | KADNVVLENGGRLDVLTGHTATNTRVDDGGTLDVRNGGTATT |
| | | VSMGNGGVLLADSGAAVSGTRSDGKAFSIGGGQADALMLEKG |
| | | SSFTLNAGDTATDTTVNGGLFTARGGTLAGTTTLNNGAILTL |
| | | SGKTVNNDTLTIREGDALLQGGSLTGNGSVEKSGSGTLTVSN |
| | | TTLTQKAVNLNEGTLTLNDSTVTTDVIAQRGTALKLTGSTVL |
| | | NGAIDPTNVTLASGATWNIPDNATVQSVVDDLSHAGQIHFTS |
| | | TRTGKFVPATLKVKNLNGQNGTISLRVRPDMAQNNADRLVID |
| | | GGRATGKTILNLVNAGNSASGLATSGKGIQVVEAINGATTEE |
| | | GAFVQGNRLQAGAFNYSLNRDSDESWYLRSENAYRAEVPLYA |
| | | SMLTQAMDYDRIVAGSRSHQTGVNGENNSVRLSIQGGHLGHD |
| | | NNGGIARGATPESSGSYGFVRLEGDLMRTEVAGMSVTAGVYG |
| | | AAGHSSVDVKDDDGSRAGTVRDDAGSLGGYLNLVHTSSGLWA |
| | | DIVAQGTRHSMKASSDNNDFRARGWGWLGSLETGLPFSITDN |
| | | LMLEPQLQYTWQGLSLDDGKDNAGYVKFGHGSAQHVRAGFRL |
| | | GSHNDMTFGEGTSSRAPLRDSAKHSVSELPVNWWVQPSVIRT |
| | | FSSRGDMRVGTSTAGSGMTFSPSQNGTSLDLQAGLEARVREN |
| | | ITLGVQAGYAHSVSGSSAEGYNGQATLNVT |
| 21 | TdCA-Ag43 | MKRHLNTCYRLVWNHMTGAFVVASELARARGKRGGVAVALSL |
| | construct | AKKFIGGLVGSLMIGGVALAGGGHVVKWGYVGKIGPAHWGDL |
| | (Tdcarbonic | AHEYFMCKVGKNQSPVDINSSVTIEAQLEPINFHYRDQISGE |
| | anhydrase + Ag43 | IVNNGHTIMVVPKEDNYIVVDGKKFHLKQFHFHSPSEHTVEG |
| | β-chain | KYYLLELHFVHQADDGQLAVIGVVFDRGAEHPEIAKLWKEAP |
| | translocator | EHEGKKELKSLVNMQALLPENLDYYRYSGSLTTPPCSEGVIW |
| | domain) | LFLKNPLQISEAQAEKFKKIMGFENNRPVQPVNARKILKGS |
| | MW fusion protein- | SSGSSTNVTLASGATWNIPDNATVQSVVDDLSHAGQIHFTS |
| | signal | TRTGKFVPATLKVKNLNGQNGTISLRVRPDMAQNNADRLVID |
| | peptide/SP: 79577. | GGRATGKTILNLVNAGNSASGLATSGKGIQVVEAINGATTEE |
| | 67 Da (80 kDa) | GAFVQGNRLQAGAFNYSLNRDSDESWYLRSENAYRAEVPLYA |
| | MW | SMLTQAMDYDRIVAGSRSHQTGVNGENNSVRLSIQGGHLGHD |
| | TdCA:27780.93 Da | NNGGIARGATPESSGSYGFVRLEGDLMRTEVAGMSVTAGVYG |
| | (28 kDa) | AAGHSSVDVKDDDGSRAGTVRDDAGSLGGYLNLVHTSSGLWA |
| | | DIVAQGTRHSMKASSDNNDFRARGWGWLGSLETGLPFSITDN |
| | | LMLEPQLQYTWQGLSLDDGKDNAGYVKFGHGSAQHVRAGFRL |
| | | GSHNDMTFGEGTSSRAPLRDSAKHSVSELPVNWWVQPSVIRT |
| | | FSSRGDMRVGTSTAGSGMTFSPSQNGTSLDLQAGLEARVREN |
| | | ITLGVQAGYAHSVSGSSAEGYNGQATLNVT |
| 22 | signal peptide | MKRHLNTCYRLVWNHMTGAFVVASELARARGKRGGV |
| | (red) | AVALSLA |
| 23 | Ag43 b-chain | PTNVTLASGATWNIPDNATVQSVVDDLSHAGQIHFTSTRTGKFVPATLK |
| | translocator | VKNLNGQNGTISLRVRPDMAQNNADRLVIDGGRATGKTILNLVNAGNS |
| | domain | ASGLATSGKGIQVVEAINGATTEEGAFVQGNRLQAGAFNYSLNRDSDES |
| | | WYLRSENAYRAEVPLYASMLTQAMDYDRIVAGSRSHQTGVNGENNSV |
| | | RLSIQGGHLGHDNNGGIARGATPESSGSYGFVRLEGDLMRTEVAGMSVT |
| | | AGVYGAAGHSSVDVKDDDGSRAGTVRDDAGSLGGYLNLVHTSSGLWA |
| | | DIVAQGTRHSMKASSDNNDFRARGWGWLGSLETGLPFSITDNLMLEPQL |
| | | QYTWQGLSLDDGKDNAGYVKFGHGSAQHVRAGFRLGSHNDMTFGEGT |
| | | SSRAPLRDSAKHSVSELPVNWWVQPSVIRTFSSRGDMRVGTSTAGSGMT |
| | | FSPSQNGTSLDLQAGLEARVREN |
| | | ITLGVQAGYAHSVSGSSAEGYNGQATLNVT |
| 24 | pSyn6-TdCA-Ag43 | CTGGTTGGCTTGGTTTCATCAGCCATCCGCTTGCCCTCATCTGTTACGCCGGCGGTAG |
| | pSyn_6 plasmid | CCGGCCAGCCTCGCAGAGCAGGATTCCCGTTGAGCACCGCCAGGTGCGAATAAGGG |
| | vector | ACAGTGAAGAAGGAACACCCGCTCGCGGGTGGGCCTACTTCACCTATCCTGCCCGGC |
| | DNA (codon | TGACGCCGTTGGATACACCAAGGAAAGTCTACACGAACCCTTTGGCAAAATCCTGTA |
| | optimized for | TATCGTGCGAAAAAGGATGGATATACCGAAAAAATCGCTATAATGACCCCGAAGCA |
| | Synechococcus | GGGTTATGCAGCGGAAGATCGATAATCTCATGACCAAAATCCCTTAACGTGAGTTTT |
| | elongatus strain | CGTTCCACTGAGCGTCAGACCCCGTATAAAAGATCAAAGGATCTTCTTGAGATCCTTT |
| | UTEX 2973) | TTTTCTGCGCGTAATCTGCTGCTTGCAAACAAAAAAACCACCGCTACCAGCGGTGGTT |
| | 6795 nucleotides | TGTTTGCCGGATCAAGAGCTACCAACTCTTTTTCCGAAGGTAACTGGCTTCAGCAGA |
| | including 2328 | GCGCAGATACCAAATACTGTTCTTCTAGTGTAGCCGTAGTTAGGCCACCACTTCAAGA |
| | nucleotides | ACTCTGTAGCACCGCCTACATACCTCGCTCTGCTAATCCTGTTACCAGTGGCTGCTGC |
| | corresponding to | CAGTGGCGATAAGTCGTGTCTTACCGGGTTGGACTCAAGACGATAGTTACCGGATAA |
| | SEQ 1 | GGCGCAGCGGTCGGGCTGAACGGGGGGTTCGTGCACACAGCCCAGCTTGGAGCGA |
| | | ACGACCTACACCGAACTGAGATACCTACAGCGTGAGCTATGAGAAAGCGCCACGCTT |
| | | CCCGAAGGGAGAAAGGCGGACAGGTATCCGGTAAGCGGCAGGGTCGGAACAGGA |
| | | GAGCGCACGAGGGAGCTTCCAGGGGGAAACGCCTGGTATCTTTATAGTCCTGTCGG |
| | | GTTTCGCCACCTCTGACTTGAGCGTCGATTTTTGTGATGCTCGTCAGGGGGGCGGAG |
| | | CCTATGGAAAAACGCCAGCAACGCGGCCTTTTTACGGTTCCTGGCCTTTTGCTGGCCT |
| | | TTTGCTCACATGTGTGCTGGGCCCCAATGCCTTCTCCAAGGGCGGCATTCCCCTGACT |
| | | GTTGAAGGCGTTGCCAATATCAAGATTGCTGGGGAAGAACCGACCATCCACAACGC |
| | | GATCGAGCGGCTGCTTGGCAAAAACCGTAAGGAAATCGAGCAAATTGCCAAGGAGA |
| | | CCCTCGAAGGCAACTTGCGTGGTGTTTTAGCCAGCCTCACGCCGGAGCAGATCAACG |
| | | AGGACAAAATTGCCTTTGCCAAAAGTCTGCTGGAAGAGGCGGAGGATGACCTTGAG |
| | | CAGCTGGGTCAAGTCCTCGATACGCTGCAAGTCCAGAACATTTCCGATGAGGTCGGT |
| | | TATCTCTCGGCTAGTGGACGCAAGCAGCGGGCTGATCTGCAGCGAGATGCCCGAATT |
| | | GCTGAAGCCGATGCCCAGGCTGCCTCTGCGATCCAAACGGCCGAAAATGACAAGAT |
| | | CACGGCCCTGCGTCGGATCGATCGCGATGTAGCGATCGCCCAAGCCGAGGCCGAGC |
| | | GCCGGATTCAGGATGCGTTGACGCGGCGCGAAGCGGTGGTGGCCGAAGCTGAAGC |
| | | GGACATTGCTACCGAAGTCGCTCGTAGCCAAGCAGAACTCCCTGTGCAGCAGGAGC |
| | | GGATCAAACAGGTGCAGCAGCAACTTCAAGCCGATGTGATCGCCCCAGCTGAGGCA |
| | | GCTTGTAAACGGGCGATCGCGGAAGCGCGGGGGGCCGCCGCCCGTATCGTCGAAG |
| | | ATGGAAAAGCTCAAGCGGAAGGGACCCAACGGCTGGCGGAGGCTTGGCAGACCGC |
| | | TGGTGCTAATGCCCGCGACATCTTCCTGCTCCAGAAGTCTAGATAATCCCTAGCGATC |
| | | GCAAGTCCAAAGGTTGTCTACAATCAATATCCAAGCATCAAAAAGCGCCCCATTCGA |
| | | GGCGCTTTTTGATTATTCAGACTGCTGTAATTCCGGCAATTAGGTTATTTGCCGACTA |
| | | CCTTGGTGATCTCGCCTTTCACGTAGTGGACAAATTCTTCCAACTGATCTGCGCGCGA |
| | | GGCCAAGCGATCTTCTTCTTGTCCAAGATAAGCCTGTCTAGCTTCAAGTATGACGGGC |
| | | TGATACTGGGCCGGCAGGCGCTCCATTGCCCAGTCGGCAGCGACATCCTTCGGCGCG |
| | | ATTTTGCCGGTTACTGCGCTGTACCAAATGCGGGACAACGTAAGCACTACATTTCGCT |
| | | CATCGCCAGCCCAGTCGGGCGGCGAGTTCCATAGCGTTAAGGTTTCATTTAGCGCCT |
| | | CAAATAGATCCTGTTCAGGAACCGGATCAAAGAGTTCCTCCGCCGCTGGACCTACCA |
| | | AGGCAACGCTATGTTCTCTTGCTTTTGTCAGCAAGATAGCCAGATCAATGTCGATCGT |
| | | GGCTGGCTCGAAGATACCAGCAAGAATGTCATTGCGCTGCCATTCTCCAAATTGCAG |
| | | TTCGCGCTTAGCTGGATAACGCCACGGAATGATGTCGTCGTGCACAACAATGGTGAC |
| | | TTCTACAGCGCGGAGAATCTCGCTCTCTCCAGGGGAAGCCGAAGTTTCCAAAAGGTC |
| | | GTTGATCAAAGCTCGCCGCGTTGTTTCATCAAGCCTTACGGTCACCGTAACCAGCAAA |
| | | TCAATATCACTGTGTGGCTTCAGGCCGCCATCCACTGCGGAGCCGTACAAATGTACG |
| | | GCCAGCAACGTCGGTTCGAGATGGCGCTCGATGACGCCAACTACCTCTGATAGTTGA |
| | | GTCGATACTTCGGCGATCACCGCTTCCCTCATAATGTTTAACTTTGTTTTAGGGCGACT |
| | | GCCCTGCTGCGTAACATCGTTGCTGCTCCATAACATCAAACATCGACCCACGGCGTAA |
| | | CGCGCTTGCTGCTTGGATGCCCGAGGCATAGACTGTACCCCAAAAAAACAGTCATAA |
| | | CAAGCCATGAAAACCGCCACTGCGCCGTTACCACCGCTGCGTTCGGTCAAGGTTCTG |
| | | GACCAGTTGCGTGAGCGCATACGCTACTTGCATTACAGCTTACGAACCGAACAGGCT |
| | | TATGTCCACTGGGTTCGTGCCTTCATCCGTTTCCACGGTGTGCGTCACCCGGCAACCT |
| | | TGGGTAGCAGCGAAGTCGAGGCATTTCTGTCCTGGCTGGCTATTTAGCGTCTTCTAAT |
| | | CCAGTGTAGACAGTAGTTTTGGCTCCGTTGAGCACTGTAGCCTTGGGCGATCGCTCT |
| | | AAACATTACATAAATTCACAAAGTTTTCGTTACATAAAAATAGTGTCTACTTAGCTAA |
| | | AAATTAAGGGTTTTTTACACCTTTTTGACAGTTAATCTCCTAGCCTAAAAAGCAAGAG |
| | | TTTTTAACTAAGACTCTTGCCCTTTACAACCTCGAAGGAGCGTCAGATCTCATATGAA |
| | | ACGCCACCTGAACACCTGCTACCGCCTGGTGTGGAACCACATGACCGGCGCCTTTGT |
| | | GGTGGCCAGCGAACTGGCCCGCGCCCGCGGCAAACGCGGCGGCGTGGCCGTGGCC |
| | | CTGAGCCTGGCCGCCGTGACCAGCCTGCCCGTGCTGGCCGGCGGCGGCCACGTGGT |
| | | GAAATGGGGCTACGTGGGCAAAATCGGCCCCGCCCACTGGGGCGATCTGGCCCACG |
| | | AATACTTTATGTGCAAAGTGGGCAAAAACCAGAGCCCCGTGGATATCAACAGCAGC |
| | | GTGACCATCGAAGCCCAGCTGGAACCCATCAACTTTCACTACCGCGATCAGATCAGC |
| | | GGCGAAATCGTGAACAACGGCCACACCATCATGGTGGTGCCCAAAGAAGATAACTA |
| | | CATCGTGGTGGATGGCAAAAAATTTCACCTGAAACAGTTTCACTTTCACAGCCCCAGC |
| | | GAACACACCGTGGAAGGCAAATACTACCTGCTGGAACTGCACTTTGTGCACCAGGCC |
| | | GATGATGGCCAGCTGGCCGTGATCGGCGTGGTGTTTGATCGCGGCGCCGAACACCC |
| | | CGAAATCGCCAAACTGTGGAAAGAAGCCCCCGAACACGAAGGCAAAAAAGAACTGA |
| | | AAAGCCTGGTGAACATGCAGGCCCTGCTGCCCGAAAACCTGGATTACTACCGCTACA |
| | | GCGGCAGCCTGACCACCCCCCCCTGCAGCGAAGGCGTGATCTGGCTGTTTCTGAAAA |
| | | ACCCCCTGCAGATCAGCGAAGCCCAGGCCGAAAAATTTAAAAAAATCATGGGCTTTG |
| | | AAAACAACCGCCCCGTGCAGCCCGTGAACGCCCGCAAAATCCTGAAAGGCAGCAGC |
| | | AGCGGCAGCAGCCCCACCAACGTGACCCTGGCCAGCGGCGCCACCTGGAACATCCC |
| | | CGATAACGCCACCGTGCAGAGCGTGGTGGATGATCTGAGCCACGCCGGCCAGATCC |
| | | ACTTTACCAGCACCCGCACCGGCAAATTTGTGCCCGCCACCCTGAAAGTGAAAAACC |
| | | TGAACGGCCAGAACGGCACCATCAGCCTGCGCGTGCGCCCCGATATGGCCCAGAAC |
| | | AACGCCGATCGCCTGGTGATCGATGGCGGCCGCGCCACCGGCAAAACCATCCTGAA |
| | | CCTGGTGAACGCCGGCAACAGCGCCAGCGGCCTGGCCACCAGCGGCAAAGGCATCC |
| | | AGGTGGTGGAAGCCATCAACGGCGCCACCACCGAAGAAGGCGCCTTTGTGCAGGGC |
| | | AACCGCCTGCAGGCCGGCGCCTTTAACTACAGCCTGAACCGCGATAGCGATGAAAG |
| | | CTGGTACCTGCGCAGCGAAAACGCCTACCGCGCCGAAGTGCCCCTGTACGCCAGCAT |
| | | GCTGACCCAGGCCATGGATTACGATCGCATCGTGGCCGGCAGCCGCAGCCACCAGA |
| | | CCGGCGTGAACGGCGAAAACAACAGCGTGCGCCTGAGCATCCAGGGCGGCCACCTG |
| | | GGCCACGATAACAACGGCGGCATCGCCCGCGGCGCCACCCCCGAAAGCAGCGGCAG |
| | | CTACGGCTTTGTGCGCCTGGAAGGCGATCTGATGCGCACCGAAGTGGCCGGCATGA |
| | | GCGTGACCGCCGGCGTGTACGGCGCCGCCGGCCACAGCAGCGTGGATGTGAAAGAT |
| | | GATGATGGCAGCCGCGCCGGCACCGTGCGCGATGATGCCGGCAGCCTGGGCGGCTA |
| | | CCTGAACCTGGTGCACACCAGCAGCGGCCTGTGGGCCGATATCGTGGCCCAGGGCA |
| | | CCCGCCACAGCATGAAAGCCAGCAGCGATAACAACGATTTTCGCGCCCGCGGCTGG |
| | | GGCTGGCTGGGCAGCCTGGAAACCGGCCTGCCCTTTAGCATCACCGATAACCTGATG |
| | | CTGGAACCCCAGCTGCAGTACACCTGGCAGGGCCTGAGCCTGGATGATGGCAAAGA |
| | | TAACGCCGGCTACGTGAAATTTGGCCACGGCAGCGCCCAGCACGTGCGCGCCGGCT |
| | | TTCGCCTGGGCAGCCACAACGATATGACCTTTGGCGAAGGCACCAGCAGCCGCGCCC |
| | | CCCTGCGCGATAGCGCCAAACACAGCGTGAGCGAACTGCCCGTGAACTGGTGGGTG |
| | | CAGCCCAGCGTGATCCGCACCTTTAGCAGCCGCGGCGATATGCGCGTGGGCACCAG |
| | | CACCGCCGGCAGCGGCATGACCTTTAGCCCCAGCCAGAACGGCACCAGCCTGGATCT |
| | | GCAGGCCGGCCTGGAAGCCCGCGTGCGCGAAAACATCACCCTGGGCGTGCAGGCCG |
| | | GCTACGCCCACAGCGTGAGCGGCAGCAGCGCCGAAGGCTACAACGGCCAGGCCACC |
| | | CTGAACGTGACCTTTTAAcatatgCACCACCACCATCACCACGAAAACCTGTACTTTCAG |
| | | GGCAAGCTTCGAATTCCCGGATCCGCGGTACCAGGCAAACCCATCCCCAACCCCCTG |
| | | CTGGGCCTGGATAGCACCGGTGGTGGTCACCACCACCATCACCACTAGAGTACTGTA |
| | | TGCATCGAGTGCCTGGCGGCAGTAGCGCGGTGGTCCCACCTGACCCCATGCCGAACT |
| | | CAGAAGTGAAACGCCGTAGCGCCGATGGTAGTGTGGGGTCTCCCCATGCGAGAGTA |
| | | GGGAACTGCCAGGCATCAAATAAAACGAAAGGCTCAGTCGAAAGACTGGGCCTTCT |
| | | CGAGTCCCTGCTCGTCACGCTTTCAGGCACCGTGCCAGATATCGACGTGGAGTCGAT |
| | | CACTGTGATTGGCGAAGGGGAAGGCAGCGCTACCCAAATCGCTAGCTTGCTGGAGA |
| | | AGCTGAAACAAACCACGGGCATTGATCTGGCGAAATCCCTACCGGGTCAATCCGACT |
| | | CGCCCGCTGCGAAGTCCTAAGAGATAGCGATGTGACCGCGATCGCTTGTCAAGAATC |
| | | CCAGTGATCCCGAACCATAGGAAGGCAAGCTCAATGCTTGCCTCGTCTTGAGGACTA |
| | | TCTAGATGTCTGTGGAACGCACATTTATTGCCATCAAGCCCGATGGCGTTCAGCGGG |
| | | GTTTGGTCGGTACGATCATCGGCCGCTTTGAGCAAAAAGGCTTCAAACTGGTGGGCC |
| | | TAAAGCAGCTGAAGCCCAGTCGCGAGCTGGCCGAACAGCACTATGCTGTCCACCGC |
| | | GAGCGCCCCTTCTTCAATGGCCTCGTCGAGTTCATCACCTCTGGGCCGATCGTGGCG |
| | | ATCGTCTTGGAAGGCGAAGGCGTTGTGGCGGCTGCTCGCAAGTTGATCGGCGCTAC |
| | | CAATCCGCTGACGGCAGAACCGGGCACCATCCGTGGTGATTTTGGTGTCAATATTGG |
| | | CCGCAACATCATCCATGGCTCGGATGCAATCGAAACAGCACAACAGGAAATTGCTCT |
| | | CTGGTTTAGCCCAGCAGAGCTAAGTGATTGGACCCCCACGATTCAACCCTGGCTGTA |
| | | CGAATAAGGTCTGCATTCCTTCAGAGAGACATTGCCATGCCG |
| 25 | Linker | G₄SGGS(G₄S)₃ |

What is claimed is:

1. An engineered outer membrane vesicle (OMV) comprising a heterologous enzyme presented on the outer surface of the engineered OMV.

2. The engineered OMV of claim 1, wherein the engineered OMV is obtained from a photosynthetic microorganism.

3. The engineered OMV of claim 2, wherein the photosynthetic microorganism is an engineered photosynthetic organism.

4. The engineered OMV of claim 2, wherein the photosynthetic microorganism is a gram-negative bacterium or eukaryotic microalgae.

5. The engineered OMV of claim 4, wherein the gram-negative bacterium is a cyanobacterium selected from Synechocystis sp., Synechococcus sp., Microcystis aeruginosa, Leptolyngbya boryana, Cyanobium gracile, Phormidium persicinum, and Gloeocapsa sp.

6. The engineered OMV of claim 1, wherein the heterologous enzyme is operably linked to a transmembrane protein embedded in the membrane of the engineered OMV to form a fusion protein.

7. The engineered OMV of claim 6, wherein the transmembrane protein is derived from an:
autotransporter protein (ATP) selected from antigen 43 (Ag43), hemoglobin-binding protease (Hbp), pertactin, extracellular serine protease plasmid-encoded (EspP), IgA1 protease, esterase autotransporter (EstA), adhesion and penetration protein (Hap), adhesin involved in diffuse adherence (AIDA-I), plasmid-encoded toxin (Pet), protease involved in intestinal colonization (Pic), temperature-sensitive hemagglutinin (Tsh), shigella extracellular protein (SepA), and vacuolating cytotoxin A (VacA); or
OMP selected from Synechococcus outer membrane protein A (SomA), MipA, and ice nucleation proteins (INPs).

8. The engineered OMV of claim 7, wherein the transmembrane protein comprises the amino acid sequence of any one of SEQ ID NOs: 8-12.

9. The engineered OMV of claim 6, wherein the fusion protein further comprises a linker connecting the heterologous enzyme to the transmembrane protein.

10. The engineered OMV of claim 1, wherein the engineered OMV comprises at least about 10 heterologous enzyme molecules presented on its outer surface.

11. The engineered OMV of claim 1, wherein the heterologous enzyme comprises a carbonic anhydrase (CA) polypeptide or functional fragment thereof.

12. The engineered OMV of claim 11, wherein the CA polypeptide is derived from a thermostable CA from Thermosulfurimonas dismutans or comprises the amino acid sequence of SEQ ID NO: 7.

13. An engineered host cell capable of producing an OMV comprising a heterologous enzyme presented on the outer surface of the OMV, wherein the engineered host cell is a photosynthetic microorganism comprising heterologous nucleic acid encoding a fusion protein comprising the heterologous enzyme operably linked to a transmembrane protein, and wherein the fusion protein is capable of being inserted into the outer membrane of the engineered host cell such that the heterologous enzyme is presented on the outer surface of the engineered host cell.

14. The engineered host cell of claim 13, wherein the transmembrane protein is derived from:
an autotransporter protein (ATP) selected from antigen 43 (Ag43), hemoglobin-binding protease (Hbp), pertactin, extracellular serine protease plasmid-encoded (EspP), IgA1 protease, esterase autotransporter (EstA), adhesion and penetration protein (Hap), adhesin involved in diffuse adherence (AIDA-I), plasmid-encoded toxin (Pet), protease involved in intestinal colonization (Pic), temperature-sensitive hemagglutinin (Tsh), shigella extracellular protein (SepA), and vacuolating cytotoxin A (VacA): or
an outer membrane protein (OMP), wherein the transmembrane protein derived from an OMP selected from Synechococcus outer membrane protein A (SomA), MipA, and ice nucleation proteins (INPs)

15. The engineered host cell of claim 14, wherein the transmembrane protein comprises the amino acid sequence selected from SEQ ID NOs: 8-12.

16. The engineered host cell of claim 13, wherein the fusion protein further comprises:
a linker connecting the heterologous enzyme to the transmembrane protein; and
a signal peptide capable of targeting the fusion protein to the inner membrane of the engineered host cell.

17. The engineered host cell of claim 13, wherein the engineered host cell is a:
cyanobacterium selected from Synechocystis sp., Synechococcus sp., Microcystis aeruginosa, Leptolyngbya boryana, Cyanobium gracile, Phormidium persicinum, and Gloeocapsa sp: or
eukaryotic microalgae.

18. The engineered host cell of claim 13, wherein the heterologous enzyme comprises a CA polypeptide or functional fragment thereof.

19. The engineered host cell of claim 13, wherein the heterologous nucleic acid is present extrachromosomally and is integrated into the chromosome of the engineered host cell.

20. A method of producing an engineered OMV comprising:
(a) culturing the engineered host cell according to claim 13 under conditions suitable for expression of the fusion protein; and
(b) obtaining an OMV produced by the engineered host cell,
thereby producing the engineered OMV.

21. The method of claim 20, wherein the engineered host cell is cultured in the presence of CO₂ and light, and wherein the method further comprises filtering the engineered OMV.

22. A nanocatalyst composition comprising the engineered OMV of claim 1.

23. The nanocatalyst composition of claim 22, comprising the engineered OMV at a concentration greater than about 100 mg/L or 10 g.

24. The nanocatalyst of claim 22, wherein the composition is not a therapeutic composition.

25. A method of reducing CO₂ comprising interacting the engineered OMV according to claim 11 with CO₂ under a condition suitable for the CA polypeptide or functional fragment thereof to convert CO₂ to a catalysis product.

26. The method of claim 25, wherein the catalysis product is a metal carbonate.

27. The method of claim 26, wherein the engineered OMV is interacted with CO₂ in the presence of H₂O and a metal ion under alkaline conditions suitable for (i) the conversion of CO₂ to CO₃²⁻ by the CA polypeptide or functional fragment thereof, and (ii) association of CO₃²⁻ with the metal ion to form the metal carbonate.

28. The method of claim 27, wherein the metal ion is Ca²⁺, Mg²⁺, Fe²⁺, Zn²⁺, Cu²⁺, Mn²⁺, or Li⁺.

29. The method of claim 25, wherein the engineered OMV is interacted with CO₂ in air, water, or waste.

30. A system for the production of an engineered outer membrane vesicle (OMV) and for catalyzing the conversion of CO₂ and H₂O to HCO₃⁻ and H⁺, the system comprising:
(a) a first reactor configured to receive a microorganism capable of producing the engineered OMV:
(b) a receptacle configured to receive the engineered OMV produced in the first reactor, wherein the receptable is capable of being in conditions suitable for carrying out the conversion of CO₂ and H₂O to HCO₃⁻ and H⁺; and
(c) a filtration interface connecting the first reactor to the receptable, wherein the engineered OMV produced in the first reactor is capable of passing through the filtration interface into the receptacle.
